(19) United States
(12) Patent Application Publication
(10) Pub. No.: US 2026/0295163 A1
(43) Pub. Date: Oct. 1, 2026

(54) Reusable automatic medicament delivery device

(71) Applicant: SHL Medical AG, Zug (CH)

(72) Inventors: Daniel Carlsson, Nacka Strand (SE); Daniel Säll, Nacka Strand (SE)

(21) Appl. No.: 19/478,039

(22) PCT Filed: Apr. 12, 2024
(86) PCT No.: PCT/EP2024/059959

§ 371 Date: Oct. 23, 2025

Related U.S. Application Data
(60) Provisional application No. 63/462,557, filed on Apr. 28, 2023.

(30) Foreign Application Priority Data
Jul. 18, 2023 (EP) 23186212.9

(51) Int. Cl. A61M 5/20; A61M 5/28; A61M 5/315; A61M 5/32

(52) U.S. Cl. CPC A61M 5/2033; A61M 5/28; A61M 5/31501; A61M 5/31578; A61M 5/3202; A61M 5/3245; A61M 2005/2013; A61M 2005/206; A61M 2005/3247; A61M 2205/121; A61M 2205/273

(57) ABSTRACT

The present disclosure provides a two-part medicament delivery device with which a user may connect a disposable cassette subassembly to a reusable drive mechanism subassembly. Connection of the subassemblies sets (or resets) and cocks a plunger rod within the reusable drive mechanism subassembly. The plunger rod has a hollow portion that accepts a drive spring that is compressed during connection of the subassemblies. The plunger rod is locked and fixed axially relative to outer housing of the drive mechanism subassembly when connection is complete. Axial distal movement of a needle guard pushes an activation sleeve distally unlocking the piston rod so that it moves proximally and dispenses medicament from a medicament container in the disposable cassette subassembly.

### TECHNICAL FIELD

The present disclosure is directed to a medicament delivery device, and particularly but not exclusively to a two-piece automatic medicament delivery device where a disposable cassette subassembly containing a medicament container may be removably connected to a reusable drive mechanism subassembly. Connection of the disposable cassette subassembly to the drive mechanism subassembly loads and cocks a drive mechanism prior to user activation of the delivery device when a needle guard slidably positioned with the disposable cassette subassembly is pushed against an injection site.

### BACKGROUND

Injection devices, such as auto-injectors, that are configured with a disposable medicament container part and a reusable part are known. For example, U.S. Pat. No. 10,599,309 relates to medicament delivery devices that are available as and delivered to a user as two connectable subassemblies for final assembly. One of the subassemblies may be a powerpack, which requires that the proximal end of the other subassembly to be removably inserted into the powerpack to tension and set a driver. A trigger on the powerpack is then activated to cause the driver to engage a plunger rod that is part of the medicament container in the second subassembly. The driver moves the medicament container proximally until the injection needle exits the proximal end of the housing of the second subassembly and penetrates the injection site. The driver then pushes on the plunger rod to administer the medicament. The setting of the driver and the requirement of a moving medicament container during activation are drawbacks of this known two-piece medicament delivery device.

### SUMMARY

In the present disclosure, when the term “distal” is used, this refers to the direction pointing away from the dose delivery site. When the term “distal part/end” is used, this refers to the part/end of the delivery device, or the parts/ends of the members thereof, which under use of the medicament delivery device is/are located furthest away from the dose delivery site. Correspondingly, when the term “proximal” is used, this refers to the direction pointing to the dose delivery site. When the term “proximal part/end” is used, this refers to the part/end of the delivery device, or the parts/ends of the members thereof, which under use of the medicament delivery device is/are located closest to the dose delivery site.

Further, the term “longitudinal”, with or without “axis”, refers to a direction or an axis through the device or components thereof in the direction of the longest extension of the device or the component.

The term “lateral”, with or without “axis”, refers to a direction or an axis through the device or components thereof in the direction of the broadest extension of the device or the component. “Lateral” may also refer to a position to the side of a “longitudinally” elongated body.

In a similar manner, the terms “radial” or “transversal”, with or without “axis”, refers to a direction or an axis through the device or components thereof in a direction generally perpendicular to the longitudinal direction, e.g. “radially outward” would refer to a direction pointing away from the longitudinal axis.

Also, if nothing else is stated, in the following description wherein the mechanical structure of the device and the mechanical interconnection of its components is described, the device is in an initial non-activated or non-operated state.

In view of the foregoing, a general object of the present disclosure is to provide a two-part medicament injection device that is assembled by a user through the connection of a reusable drive mechanism subassembly with a disposable cassette assembly. The disposable cassette subassembly automatically sets or cocks a plunger rod in the reusable drive mechanism subassembly such that when the assembled injection device is activated, the cocked and locked plunger rod will be released to engage a sliding piston in a medicament container positioned in the disposable cassette subassembly to dispense medicament to an injection site. Once the medicament has been dispensed, the used disposable cassette subassembly is removed from the reusable drive mechanism subassembly and may be discarded. Connecting a new disposable cassette subassembly resets the plunger rod.

The reusable drive mechanism subassembly may include a tubular housing having an open proximal end and a closed distal end, a plunger rod having a proximal portion having a first diameter and a distal portion having a second diameter that is larger than the first diameter, a drive spring partially positioned in a hollow section of the distal portion, an activation sleeve partially surrounding the plunger rod and slidable relative to the tubular housing, and a plunger rod lock mechanism that may include a radially biased plunger rod lock pivotally attached to the housing such that the plunger rod lock operatively engages with the plunger rod and the activation sleeve.

The disposable cassette subassembly may have a cassette body, a cap removably attached to the cassette body, a needle guard partially positioned inside and slidable relative to the cassette body, where the cassette body rotates relative to the needle guard when the disposable cassette subassembly is operatively connected to the drive mechanism subassembly, a plunger rod interface positioned at a distal end of the cassette body such that the cassette body can rotate relative to the plunger rod interface, and a medicament container having an attached needle. Such an embodiment may be configured such that the plunger rod interface engages with the proximal portion of the plunger rod when the disposable cassette subassembly is connected to the drive mechanism subassembly pushing the plunger rod distally until the plunger rod lock mechanism transitions from a released position to a locked position such that the plunger rod is prevented from proximal axial movement relative to the medicament container.

The above-described reusable drive mechanism subassembly may further include an activation sleeve spring that biases the activation sleeve in a proximal direction relative to the tubular housing. The cassette body of the disposable cassette subassembly may have a locking hub protruding radially from an outside surface and configured to form a releasable bayonet attachment with an inside surface of the tubular housing. A distally projecting tab on the needle guard may engage a proximal end of the activation sleeve to move the activation sleeve distally during activation of the injection device. The needle guard is preferably rotationally fixed relative to the activation sleeve when the disposable cassette subassembly is operatively connected to the reusable drive mechanism subassembly.

The needle guard may be in an axially fixed state relative to the cassette body prior to the rotation of cassette body relative to the needle guard. Rotation of the cassette body during connection of the disposable cassette subassembly to the drive mechanism subassembly may transition the needle guard to a distally movable state such that the needle guard may engage with and move the activation sleeve distally. Distal movement of the activation sleeve may cause the activation sleeve to engage the plunger rod lock moving the plunger rod lock from a locked position to a released position releasing the plunger rod to move proximally relative to the medicament container to dispense medicament. Connection of the two subassemblies may cause ribs on an inside surface of the plunger rod interface to engage with a bearing surface on the proximal portion of the plunger rod to push the plunger rod distally to the cocked and locked state.

According to another aspect of the present disclosure there is provided a two-part medicament injection device having a reusable drive mechanism subassembly including a tubular housing having an open proximal end and a closed distal end, a plunger rod comprising a proximal portion having a first diameter and a distal portion having a second diameter that is larger than the first diameter, a drive spring partially positioned in a hollow section of the distal portion, an activation sleeve partially surrounding the plunger rod and slidable relative to the tubular housing, and a plunger rod lock mechanism with a radially biased plunger rod lock pivotally attached to the housing such that the plunger rod lock operatively engages with the plunger rod and the activation sleeve. The disposable cassette subassembly in this second embodiment may have a cassette body having a distal end with a flexible finger, a cap removably attached to the cassette body, a needle guard partially positioned inside and slidable relative to the cassette body, where the needle guard rotates relative to the cassette body when moving from a retracted position to an extended position and a medicament container comprising an attached needle.

This second embodiment is further characterized in that the flexible finger may engage with the proximal portion of the plunger rod when the disposable cassette subassembly is axially inserted into and connected to the drive mechanism subassembly such that the plunger rod is pushed distally until the plunger rod lock mechanism transitions from a released position to a locked position to prevent proximal axial movement of the plunger rod relative to the medicament container. The reusable drive mechanism subassembly may further include a cassette interface that is rotatable relative to the tubular housing.

The rotatable cassette interface of the second embodiment may have a first longitudinal channel that is slidably engaged with the flexible finger to bias the finger radially inward to engage the proximal portion of the plunger rod. Additionally, the cassette interface may have a second longitudinal channel that aligns and becomes slidably engaged with the flexible finger when a release lever is moved to overcome a rotational spring bias of the cassette interface during disconnection of the disposable cassette subassembly from the reusable drive mechanism subassembly. This embodiment may also include an electronics unit having a plurality of mechanical contact sensors that determine operational status of the injection device.

Yet still another aspect of the present disclosure is drawn to a two-part medicament injection device having a reusable drive mechanism subassembly that may include a tubular housing having an open proximal end and a closed distal end, where an inner surface of the proximal end comprises a first thread, a hollow plunger rod having a closed proximal end and an open distal end, a drive spring partially positioned inside plunger rod, an activation sleeve partially surrounding the plunger rod and slidable relative to the tubular housing, where a distal portion of the activation sleeve has a cut-out aligned with a cut-out in the plunger rod prior to activation of the injection device, and an inner housing partially surrounding the activation sleeve and having a plunger rod lock mechanism comprising a flexible finger that releasably engages with the cut-out in the plunger rod.

The disposable cassette subassembly of the third embodiment may have a cassette body with an outer surface having a second thread that matches and cooperates with the first thread such that the disposable cassette subassembly may be removably connected to the drive mechanism subassembly, a cap rotationally fixed and removably attached to the cassette body, a needle guard partially positioned inside and slidable relative to the cassette body, where the cassette body is rotationally fixed relative to the needle guard, a needle guard lock that is slidable relative to the cassette body from a first position to a second position, and a medicament container comprising an attached needle.

In this third embodiment, the axial distal movement of the needle guard may move the needle guard lock from the first position to the second position and a distal end of the cassette body may engage a proximal end of the inner housing when the cassette body is being threadedly connected to the tubular housing causing the inner housing to move axially such that the flexible finger on the inner housing engages the cut-out in the plunger rod pushing the plunger rod distally and compressing the drive spring. The activation sleeve may move relative to the inner housing during activation of the injection device causing the flexible finger to disengage from the cut-out in the plunger rod.

The needle guard may have a first cut-out and a second cut-out, where locking fingers at a proximal end of the needle guard lock are engaged with the first cut-out when the needle guard lock is in the first position and in the second position. Axial proximal movement of the needle guard relative to the needle guard lock when in the second position may cause disengagement of the locking fingers from the first cut-out and then engagement with the second cut-out.

The injection device of the third embodiment may also be configured such that the closed distal end has an endcap having a longitudinal support that is positioned within the drive spring and support arms slidably engaged with an inside surface of the activation sleeve. Additionally, the protective cap may have a plurality of segmented fingers having inner surfaces configured with a snap fit connector that engages with a proximal end of the cassette body, where when the cassette body is threadedly connected to the tubular housing the segmented fingers engage a ramp surface on the cassette body causing the segmented fingers to bend radially outward disconnecting the snap fit connector from the cassette body.

### BRIEF DESCRIPTION OF THE DRAWINGS

FIG. 1 shows a perspective view of one possible embodiment of the two-piece medicament delivery device according to the present disclosure;

FIG. 2 shows an exploded view of the disposable cassette subassembly medicament delivery device of FIG. 1;

FIG. 3 shows an exploded view of the reusable drive mechanism subassembly of the medicament delivery device of FIG. 1;

FIG. 4 shows close up perspective view to the plunger rod of the disposable cassette subassembly of the medicament delivery device of FIG. 1;

FIG. 5 shows a perspective view of the disposable cassette subassembly of FIG. 1;

FIG. 6 shows an interior view of the interaction between the needle guard and the cassette body of the disposable cassette subassembly of FIG. 1;

FIG. 7 shows another interior view of the interaction between the needle guard and the cassette body of the disposable cassette subassembly of FIG. 1;

FIG. 8 shows a perspective cross-sectional view of the reusable drive mechanism subassembly of the medicament delivery device of FIG. 1;

FIG. 9 shows a perspective cross-sectional view of a portion of the reusable drive mechanism subassembly of the medicament delivery device of FIG. 1;

FIG. 10 shows a perspective cross-sectional view of another portion of the reusable drive mechanism subassembly of the medicament delivery device of FIG. 1;

FIG. 11 shows a perspective cross-sectional view of another portion of the reusable drive mechanism subassembly of the medicament delivery device of FIG. 1;

FIG. 12 shows a perspective partically cut away view of a portion of the reusable drive mechanism subassembly of the medicament delivery device of FIG. 1 before connection with the disposable subassembly;

FIG. 13 shows a perspective partially cut away view of a portion of the reusable drive mechanism subassembly of the medicament delivery device of FIG. 1 when connected to the disposable cassette subassembly;

FIG. 14 is a perspective close-up of the plunger lock of the medicament delivery device of FIG. 1;

FIGS. 15 and 16 show perspective views of selected internal components before and after connecting the disposable cassette subassembly to the reusable drive mechanism subassembly of the medicament delivery device of FIG. 1;

FIGS. 17a and 17b show cross-sectional views of selected internal components respectively before and after rotatably connecting the disposable cassette subassembly to the reusable drive mechanism subassembly of the medicament delivery device of FIG. 1;

FIGS. 18a and 18b show perspective views of selected internal components before and after rotatably connecting the disposable cassette subassembly to the reusable drive mechanism subassembly of the medicament delivery device of FIG. 1;

FIGS. 19a and 19b show partial cross-sectional views of the plunger rod lock before and after locking the plunger rod from axial movement;

FIGS. 20a and 20b show further cross-sectional views of the plunger rod lock before and after locking the plunger rod from axial movement;

FIGS. 21 and 22 show a perspective and a cross-sectional view of the protective cap connected with the cassette body of the disposable cassette subassembly of the medicament delivery device of FIG. 1;

FIGS. 23 and 24 show cross-sectional views of the medicament delivery device of FIG. 1 during activation and delivery of medicament;

FIGS. 25 and 26 show cross-sectional views of the medicament delivery device of FIG. 1 during completion of medicament delivery and removal of the needle guard from the injection site;

FIG. 27a shows a perspective cross-sectional view of the plunger rod interface during removal of the disposable cassette subassembly from the drive mechanism subassembly of the medicament delivery device of FIG. 1;

FIGS. 27b and 27c show further cut-away views of the plunger rod interface during removal of the disposable cassette subassembly from the drive mechanism subassembly of the medicament delivery device of FIG. 1;

FIGS. 28a to 28c show close of views of the plunger rod interface and orientations of the internal ribs relative to the cassette body;

FIG. 29 shows a perspective view of a second embodiment of the delivery device of the present disclosure before the disposable cassette subassembly is connected to the reusable drive mechanism subassembly;

FIG. 30 shows a perspective view of the delivery device of FIG. 29 after the disposable cassette subassembly is connected to the reusable drive mechanism subassembly;

FIG. 31 shows a cross-sectional view of the protective cap being disconnected from the drive mechanism subassembly of FIG. 29;

FIG. 32 shows an exploded view of the delivery device of FIG. 29;

FIG. 33 shows a perspective view of a housing of the delivery device of FIG. 29, showing a protrusion and support arms associated with an end cap;

FIG. 34 shows a cut away view of the disposable cassette subassembly of the delivery device of FIG. 29;

FIGS. 35a, 35b and 35c show cross-sectional views of the delivery device of FIG. 29 during three different stages of operation;

FIGS. 36a, 36b and 36c show different cross-sectional views of the three stages of operation shown in FIG. 35;

FIG. 37 shows a perspective close-up view of the plunger rod of the reusable drive mechanism subassembly of FIG. 29;

FIG. 38 shows a perspective view of an activation sleeve of the reusable drive mechanism subassembly of FIG. 29;

FIG. 39 shows a perspective partially exploded view of the interaction of several components of the delivery device of FIG. 29 during cocking of the plunger rod;

FIG. 40 shows a perspective view of a third embodiment of the delivery device of the present disclosure before the disposable cassette subassembly is connected to the reusable drive mechanism subassembly;

FIG. 41 shows an exploded view of the delivery device of FIG. 40;

FIG. 42 shows a partially cut away view of the disposable cassette subassembly of the delivery device of FIG. 40;

FIG. 43 shows a cross-sectional view of the protective cap connected to the disposable cassette subassembly of the delivery device of FIG. 40;

FIG. 44 shows a close-up perspective view of the needle guard in an extend position relative to the cassette body of the disposable cassette subassembly of FIG. 40;

FIG. 45 shows a close-up perspective view of the of the needle guard and cassette body of the disposable cassette subassembly of FIG. 40;

FIG. 46 shows a partially cut away view of the of the delivery device of FIG. 40 of the reusable drive mechanism subassembly before connection to the disposable cassette subassembly;

FIG. 47 shows a perspective close-up cut away view of a portion of the cassette interface of the disposable cassette subassembly of FIG. 40;

FIGS. 48a and 48b show perspective close-up cross-sectional views of the interaction of the cassette body with the cassette interface and plunger rod for the delivery device of FIG. 40;

FIGS. 49a and 49b show close-up cross-sectional views of the interaction of the cassette body with the cassette interface and plunger rod for the delivery device of FIG. 40;

FIG. 50 shows a cut away view of the of the delivery device of FIG. 40 when activated;

FIGS. 51-55 show electronic switch positions during different stages of operation of the delivery device of FIG. 40; and

FIGS. 56a and 56b show the cassette interface during removal of the disposable cassette subassembly from the reusable drive mechanism subassembly of FIG. 40, oriented before and after removal respectively.

### DETAILED DESCRIPTION

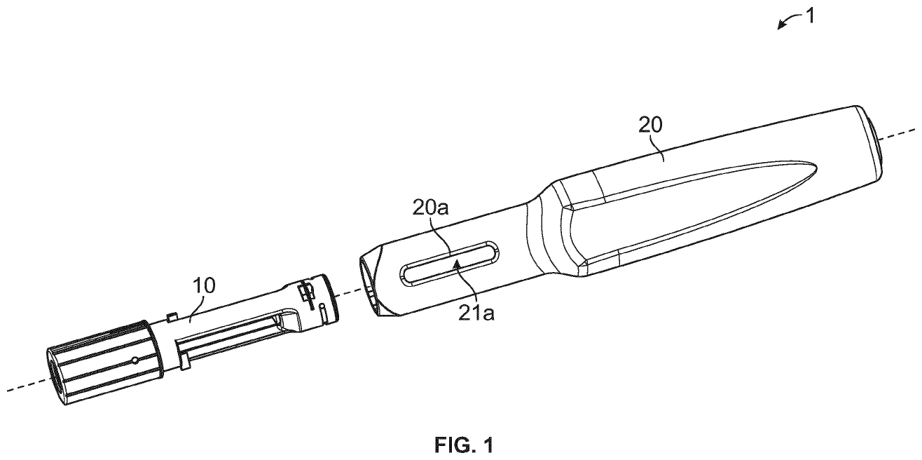

FIG. 1 shows one possible embodiment of the reusable medicament delivery device 1 of the present disclosure, where a disposable cassette subassembly 10 is separated from a reusable drive mechanism subassembly 20. A proximal portion 21a of a plunger rod 21 is visible through window (cut-out) 20a in outer housing 23.

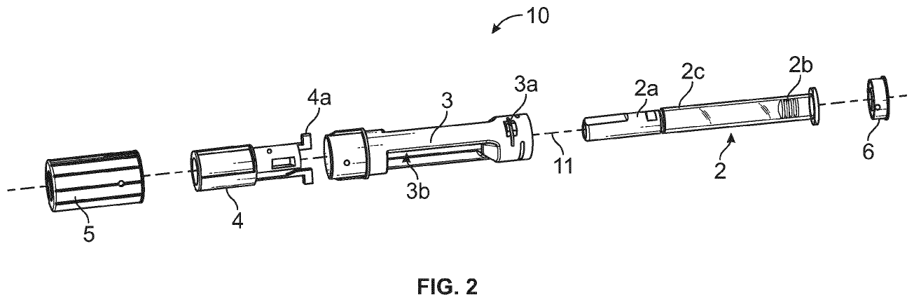

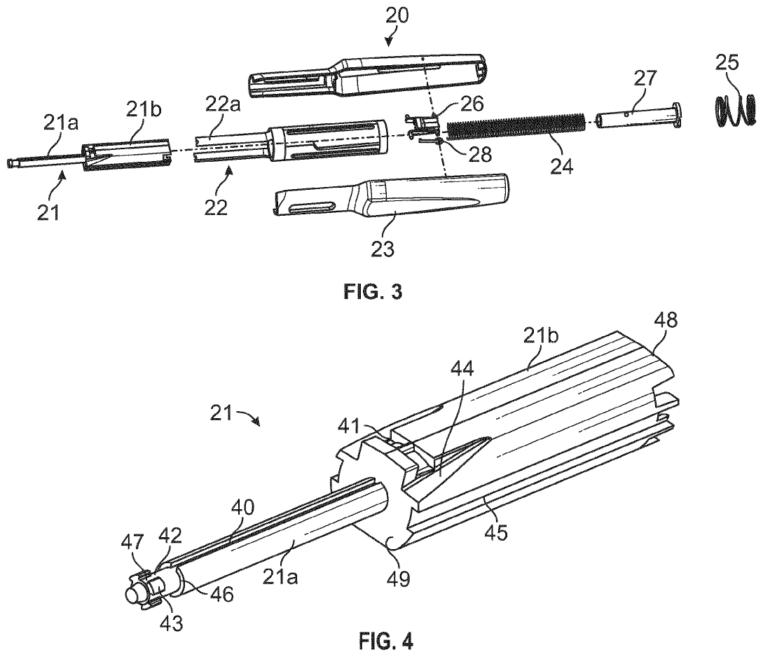

FIGS. 2 and 3 illustrate exploded perspective views of each of the two subassemblies 10, 20, respectively. The disposable cassette subassembly 10 is arranged to receive a medicament container 2. In the illustrated embodiment, the medicament container 2 is a prefilled syringe having a plunger 2b slidable in a barrel 2c and a needle shield assembly 2a covering an injection member fixed at the proximal end of the barrel 2c. The medicament container 2 is axially fixed inside and relative to a cassette body 3 of the disposable cassette assembly 10. The cassette body 3 has a locking hub 3a extending radially outward from a distal end. Although only one locking hub 3a is illustrated, preferably there are at least two such locking hubs spaced circumferentially (equally, or 180° apart in the case of just two). A needle guard 4 is at least partially contained within and slidable relative to the cassette body 3. The needle guard 4 has a radial tab 4a extending radially outward. Although only one radial tab 4a is mentioned here, preferably there are at least two such radial tabs (spaced circumferentially equally, or 180° apart in the case of just two). When the needle guard 4 is mounted to the cassette body 3, the radial tab 4a is located in a cut-out 3b of the cassette body 3 and is slidable within the cut out 3b. A protective cap 5 is releasably connected to a proximal end of the cassette body 3. The protective cap 5 is configured to remove the needle shield assembly 2a of the medicament container 2 when the cap 5 is removed from the proximal end of cassette body 3.

The disposable cassette subassembly 10 further includes a plunger rod interface 6 axially fixed to the distal end of cassette body 3. The cassette body 3 may rotate relative to the plunger rod interface 6. Rotation of the cassette body 3 may occur when a user grasps and twists/rotates the cap 5, which is rotationally fixed relative to the cassette body 3. This twisting motion occurs during insertion and connection of the disposable cassette subassembly 10 (in)to the reusable drive mechanism subassembly 20. Removal of the disposable cassette subassembly 10 after the medicament in the medicament container 2 has been administered is achieved by gripping and rotating the needle guard 4 in the opposite direction.

The reusable drive mechanism subassembly 20 comprises a housing 23, which is shown in FIG. 3 as a tubular housing with an open proximal end and a closed distal end. In the illustrated embodiment, the housing 23 is a two-part structure. The reusable drive mechanism subassembly 20 includes the plunger rod 21, an activation sleeve 22, a drive element 24, a guide rod 27, an activation sleeve biasing element 25, a plunger lock 26 and an associated lock biasing element 28. The plunger rod 21, as illustrated in FIG. 4, has the proximal portion 21a and a distal portion 21b. In the illustrated embodiment, the distal portion 21b is hollow and has an open end 48 such that it can accommodate the drive element 24 and the guide rod 27. A proximal end of the drive element 24 abuts and may be biased against an inner end wall surface of the distal portion 21b, located at a transition point 49 that defines the start of the proximal portion 21a. The distal portion 21b also has a longitudinal guide 45 on an outer surface. The longitudinal guide 45 is configured to be slidably engaged with a corresponding guide tab located on an inner surface of the housing 23 to prevent rotation of the plunger rod 21 relative to the housing 23. The proximal end of the distal portion 21b also has an inclined section 44 and cut-out 41 that are operatively associated with the plunger lock 26, as will be described below. The proximal portion 21a is shown as a shaft having at least one longitudinal slot 40 that terminates at a reduced diameter section 42 at the proximal end of the proximal portion 21a. The reduced diameter section 42 partially defines a bearing end face 46. Radial protrusions 43 are provided on the proximal portion 21b proximal to the reduced diameter section 42. The radial protrusions 43 are separated by channels 47. At least one of the channels 47 is aligned with and has a width similar to that of the longitudinal slot 40. In the illustrated embodiment there are two longitudinal slots 40 each having an aligned channel 47.

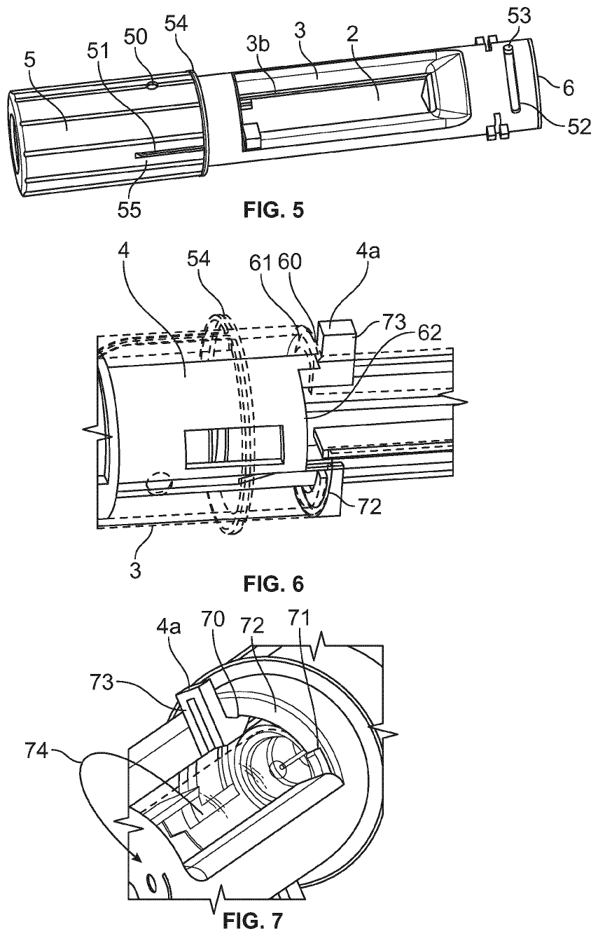

FIGS. 5-7 illustrate one possible embodiment of the structure of the disposable cassette subassembly 10 containing a pre-filled medicament container 2 as it would be received by a user prior to insertion into and connection with the reusable drive mechanism subassembly 20. The cap 5 is removably secured to the proximal end of cassette body 3. More specifically, in the illustrated embodiment, a releasable detent connector 50, shown as a radial protrusion on the cassette body 3 engageable with a hole in the cap 5, is operable to releasably secure the cap 5 to the cassette body 3. Preferably, at least two such detent connectors are provided. The cap 5 and cassette body 3 are arranged to be rotationally fixed relative to one another. An anti-rotation mechanism is provided for this purpose. More specifically, in the illustrated embodiment, the cap 5 also has a longitudinal slot 55 that accepts a correspondingly shaped longitudinal rib 51 that protrudes radially outward from the cassette body 3. This slot and rib arrangement helps to prevent rotation of the cap 5 relative to the cassette body 5. Again, preferably there are at least two such slot and rib anti-rotation engagements. When the cap 5 is releasably attached to the cassette body 3, a distally facing end wall of the cap 5 abuts a rim 54 that extends circumferentially around and radially outward from the outer surface of the cassette body 3.

As mentioned above, the needle guard 4 is slidably disposed within the cassette body 3. The radial tab 4a extends outward from the cut-out 3b. The cut-out 3b is larger than the tab 4a in both the longitudinal and circumferential directions with respect to the cassette body 3. This allows the needle guard 4 to slide axially and rotationally relative to the cassette body 3. Specifically, the cassette body 3 may rotate relative to tab 4a. Before the disposable cassette subassembly 10 is connected to the reusable drive mechanism subassembly 20, the needle guard 4 is prevented from moving axially relative to the cassette body 3 by engagement of proximal surface 60 of the radial tab 4a with cassette body distal surface 72. This interaction prevents the needle guard 4 from moving axially in the proximal direction. Likewise, distal surface 62 of the needle guard 4 engages with proximal surface 61 of the cassette body 3 to prevent distal axial movement of the needle guard 4. Rotation 74 (see FIG. 7) of cassette body 3 relative to the needle guard 4 during connection of the disposable cassette subassembly 10 to the drive mechanism subassembly 20 causes the distal surface 72 of the cassette body 3 to rotate relative to the proximal surface 60 of the radial tab 4a until the proximal surface 60 aligns with a cut-out 71 in the distal surface 72. In this orientation, the distal surface 62 of the needle guard 4 no longer engages the proximal surface 61 of the cassette body 3, with the result that the radial tab 4a and the needle guard 4 are free to move distally. This may occur when a proximal terminal end of the needle guard 4 is pushed against an injection site.

Unintentional rotation of the cassette body 3 relative to the needle guard 4 can be prevented by nib 70 that acts as a friction detent abutting the side wall of radial tab 4a. A similar anti unintentional rotation feature can be used to prevent relative rotation between the plunger rod interface 6 and the distal end of the cassette body 3. One possible anti-rotation solution is illustrated in FIG. 5 as detent 52 that includes radially projecting peg 53 on an outside surface of the plunger rod interface 6. More specifically, the cassette body 3 may have a circumferentially extending slot 52 for accommodating the peg 53 such that the angular extent of relative rotation between the plunger rod interface 6 and the cassette body 3 is limited by the circumferential length of the slot 52.

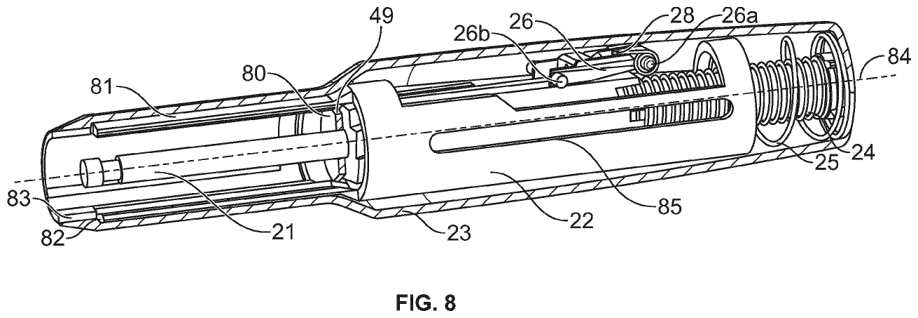

One possible embodiment of the drive mechanism subassembly 20 is illustrated in FIGS. 8-11, showing the components in a pre-cocked or a post-injection orientation, i.e. before the disposable cassette subassembly 10 is connected to or after a used disposable cassette subassembly 10 has been removed from the reusable drive mechanism subassembly 20. The plunger rod 21 is in its fully proximal position relative to the housing 23, where the transition point 49 is in abutment with web 80 extending radially inward from the inner surface of the housing 23. Likewise, the activation sleeve 22 is in its most proximal position and biased by activation sleeve biasing element 25, where the terminal proximal ends 82 of arms 81 are abutting stop ledges 83 on the inner surface of the housing 23. Plunger lock 26 is biased by lock biasing element 28 through engagement with the outer housing 23 such that the plunger lock 26 is in a sliding engagement with the outer surface of the distal portion 21b of plunger rod 21. The plunger lock 26 is axially fixed relative to housing 23, while being pivotably connected to the housing 23 through pivot pin 26a that allows the plunger lock 26 to rotate radially relative to the longitudinal axis 84 of the drive mechanism subassembly 20.

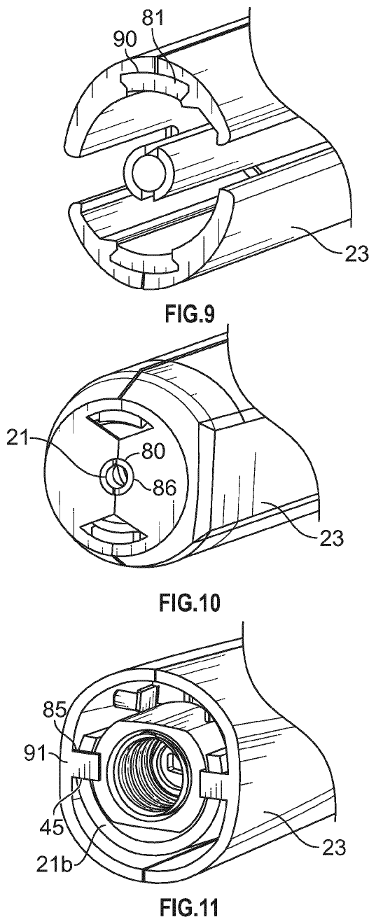

As illustrated in FIGS. 9 and 11, the activation sleeve 22 is axially guided by the housing 23 through slot 90 slidably engaged with arms 81. Similarly, cut-out 85 is guided by ribs 91 located on the inside surface of housing 23. These housing engagements with the activation sleeve 22 prevent rotation of the activation sleeve 22 relative to the housing 23. Ribs 91 also are slidably engaged with longitudinal guide 45 on the outer surface of the distal portion 21b of the plunger rod 21 (see FIGS. 4 & 11). This engagement between the plunger rod 21 and the inner surface of the housing 23 prevents rotation of the plunger rod 21. Web 80 includes a through hole 86 having a diameter that allows only the proximal portion of the plunger rod 21 to move axially relative to the housing 23 during cocking of the reusable drive mechanism 20 and during dispensing medicament from the disposable cassette subassembly 10.

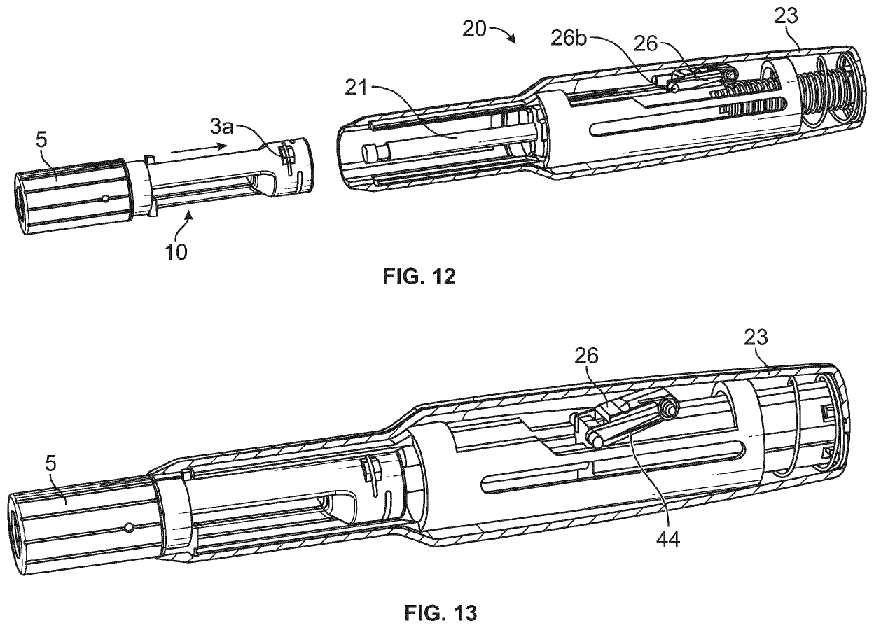

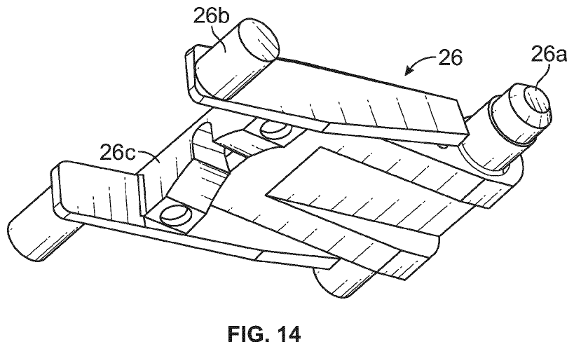

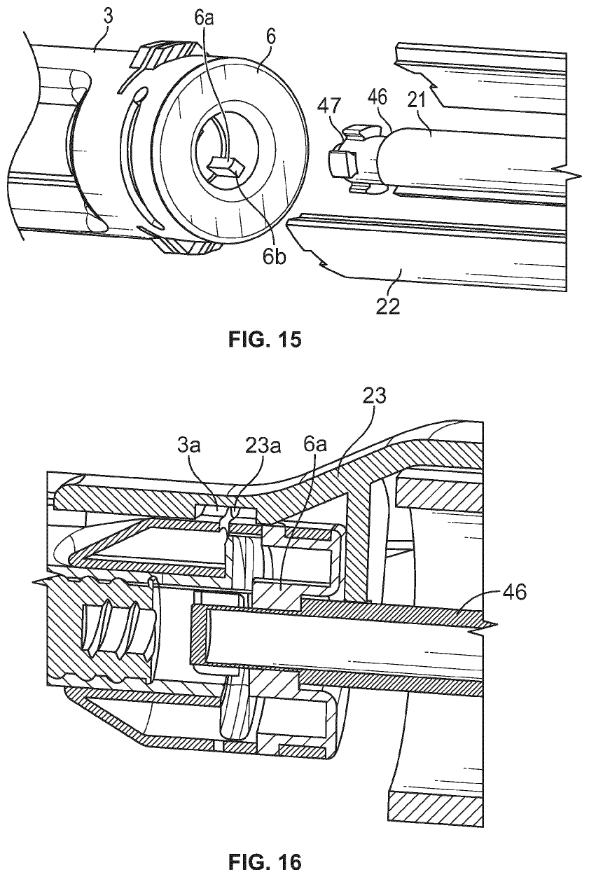

FIGS. 12 and 13 show the attachment of the disposable cassette subassembly 10 with the reusable drive mechanism subassembly 20. The radially protruding locking hub 3a is first aligned with a corresponding longitudinal slot 91 (see FIG. 17a) on the inside surface of the housing 23 and then pushed distally relative to the housing 23. This alignment of the locking hub 3a also aligns radially inward protruding ribs 6a on an inside surface of the plunger rod interface 6 with channels 47 of the plunger rod 21 such that a distally facing end face 6b of rib 6a will engage bearing end face 46 on the plunger rod 21 (see FIGS. 15 & 16). As the disposable cassette subassembly 10 is pushed into the reusable drive mechanism subassembly 20, the plunger rod interface 6 will push the plunger rod 21 distally relative to the outer housing 23 thus loading, i.e., compressing the drive element 24, to cock the drive mechanism. As the plunger rod 21 is pushed distally, the plunger lock 26 that is axially fixed to the housing 23 will slidably engage the outer surface of the distal portion 21b of the plunger rod 21. As the plunger rod 21 continues to move axially, the guide pin 26b (see FIG. 14) will eventually travel down (that is along and radially inwardly) the inclined section 44 of the plunger rod 21 until lock blade 26c drops into cut-out 41 thereby locking the plunger rod 21 from further axial movement (see FIG. 13). The lock biasing element 28 biases the plunger lock 26 to pivot in a radially inward direction maintaining contact of the plunger lock 26 with the outer surface of the plunger rod 21 and forcing the lock blade 26c into the cut-out 41.

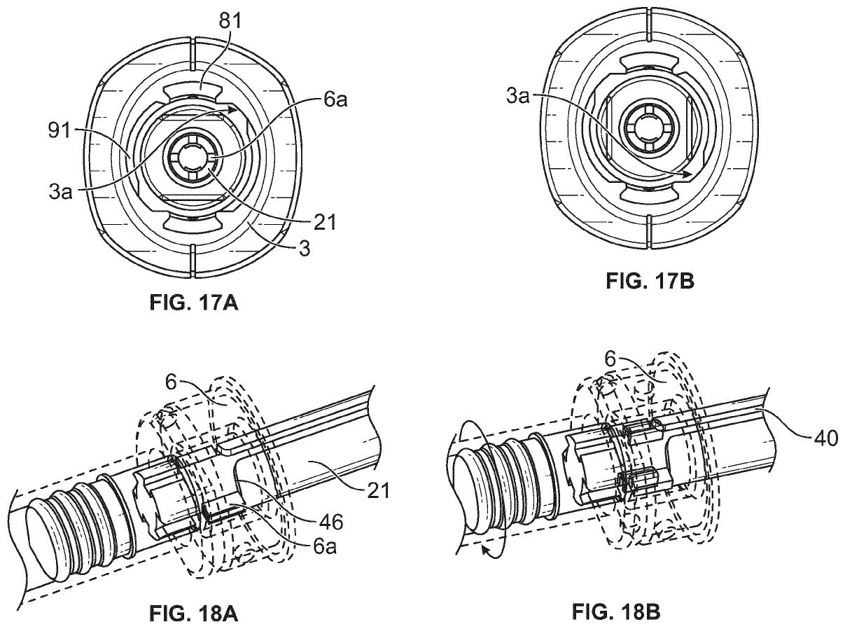

Once the plunger rod 21 is in the locked position of FIG. 13, which prevents the axial proximal movement, the user can then rotate/twist the cap 5 causing the locking hub 3a to rotate into slot 23a (see FIG. 16) forming a bayonet-type connection. This connection will prevent proximal movement of the disposable cassette subassembly 10 relative to the reusable drive mechanism subassembly 20. Such a bayonet-type connection is illustrated in FIGS. 17a and 17b in a “before” and an “after” configuration that results from inserting and twisting the cassette body 3 relative to the outer housing 23. The twisting of the disposable cassette subassembly 10 also causes the plunger rod interface 6 to rotate relative to the plunger rod 21. This rotation of the plunger rod interface 6 causes ribs 6a to move out of engagement with bearing surface 46 and to become aligned with longitudinal slot 40 (see FIGS. 18a and 18b) such that the plunger rod 21 can move axially in the proximal direction during activation of the delivery device 1 to perform the medicament delivery.

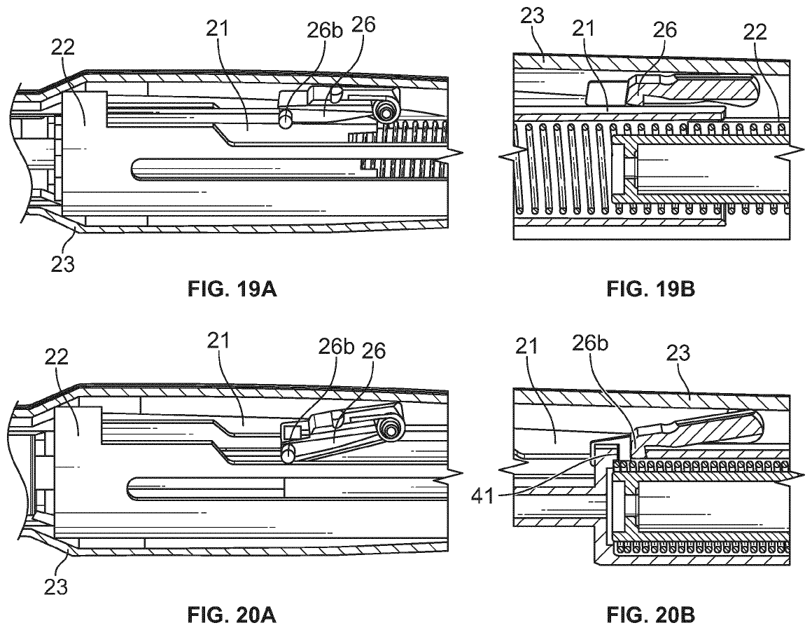

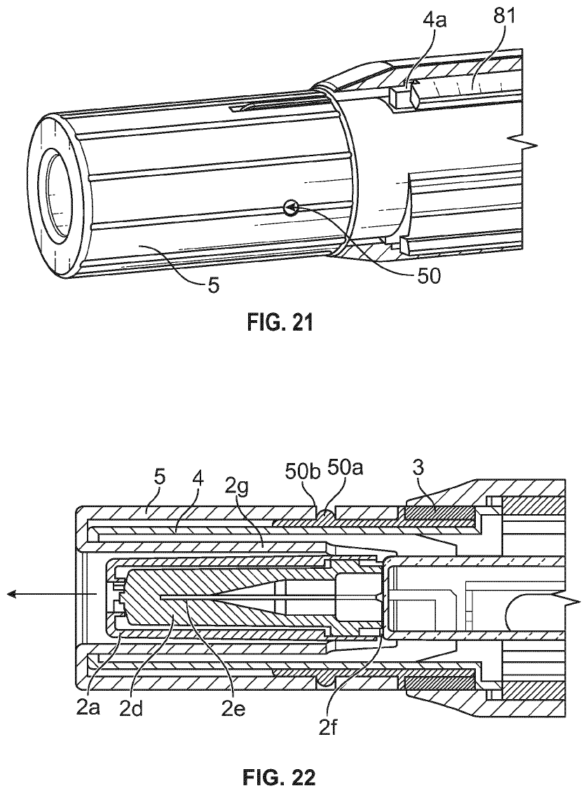

FIGS. 19a and 19b illustrate the relationship between the plunger lock 26 and the plunger rod 21 before the attachment of the disposable cassette subassembly 10 and FIGS. 20 and 20a illustrate the same relationship after the attachment. Attachment of the disposable cassette subassembly 10 moves the activation sleeve 22 distally relative to the housing 23 and slightly compresses, i.e., loads, activation sleeve biasing element 25. This movement of the activation sleeve 22 a slight distal distance is a result of the radial tab 4a engaging and pushing on the terminal end 81a of activation sleeve arm 81 of the activation sleeve 22 (see FIGS. 21 and 22). Notably, the rotation of the cassette body 3 by twisting the cap 5 does not cause the needle guard 4 to rotate because radial tab 4a is engaged with and rotationally locked to activation sleeve arms 81.

Before the fully assembled medicant delivery device 1 can be activated to deliver/administer the medicament from the medicament container 2, the cap 5 must be removed from engagement with the cassette body 3. Pulling the cap 5 axially as shown by the directional arrow in FIG. 22 will disengage detent 50, which is exemplified as a radial nib 50a on the cassette body 3 engaged with through hole 50b in the cap 5. As the cap 5 is separated from the cassette body 3, needle shield grippers 2f on the distal end of needle remover 2g engage with needle shield assembly 2a. Because needle shield remover 2g is axially fixed to cap 5, removal of the cap 5 simultaneously causes removal of shield assembly 2a, including the flexible needle shield 2d, away from needle 2e. The medicant delivery device 1 is now fully assembled and is ready to deliver a dose of medicament from the medicament container 2.

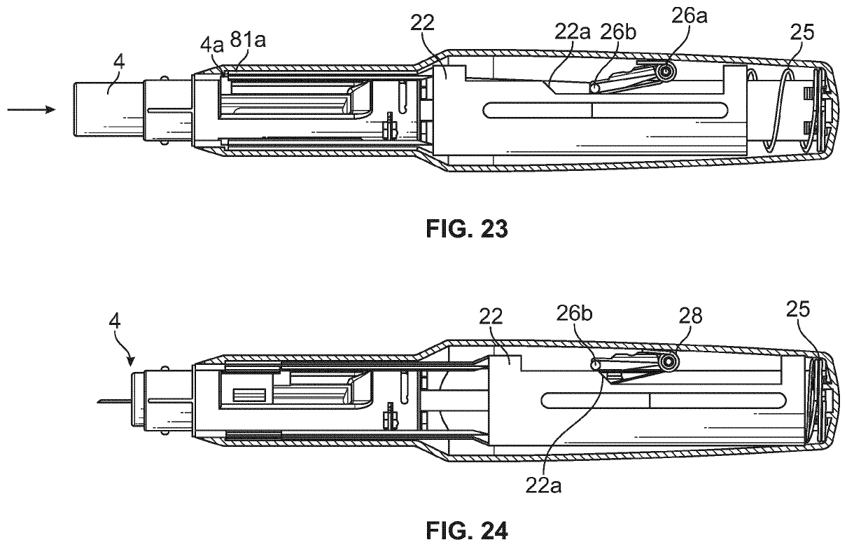

To activate the delivery device 1, the user places the terminal proximal end of needle guard 4 against an injection site and the housing 23 is pushed by the user in a proximal direction causing the needle guard 4 to retract partially into the housing 23 as indicated by the directional arrow illustrated in FIG. 23. As the needle guard 4 moves distally into the housing 23, the radial tab 4a pushes the terminal end 81a of activation sleeve arm 81 in the distal direction. As illustrated in FIG. 24, as the activation sleeve 22 moves distally, a ramp 22a on the activation sleeve 22 interacts with the guide pin 26b of the plunger lock 26 to overcome the biasing force exerted by plunger rod lock biasing element 28. This interaction causes the plunger lock 26 to pivot and rotate outward about pivot pin 26a, which is fixed the housing 23, or in the illustrated embodiment at least axially fixed to and/or positioned within an inside surface of the housing 23. As the plunger lock 26 pivots radially outward relative to the longitudinal axis of the activation sleeve 22, this causes lock blade 26c to rotate/lift out of cut-out 41 in the outer surface of plunger rod 21. Once the lock blade 26c is disengaged from the cut-out 41, the plunger rod 21 is no longer held axially fixed relative to the housing 23.

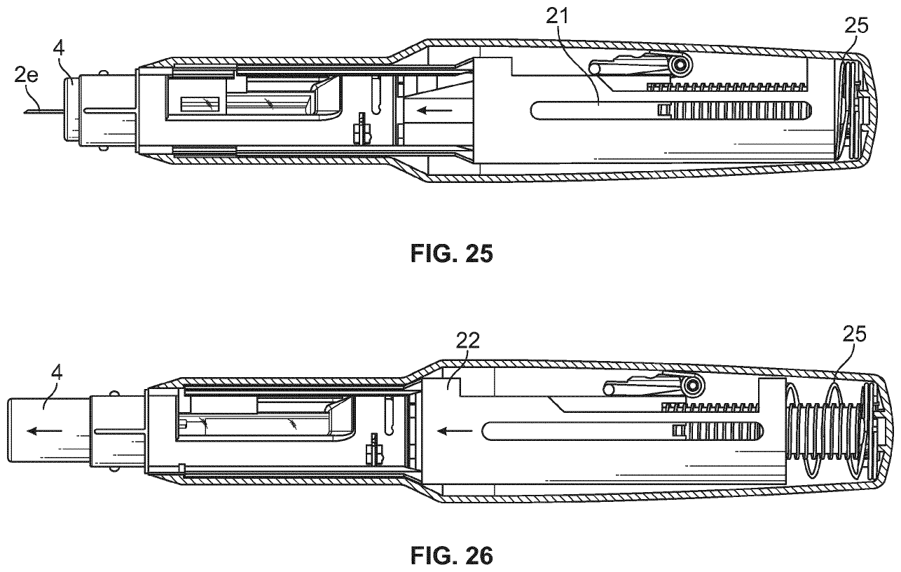

The drive element 24 (which in the illustrated embodiment is a compression spring) will then force the plunger rod 21 to move axially forward in the proximal direction as illustrated by the directional arrow in FIG. 25 (by expansion from a compressed configuration). At this point in the medicament delivery process, the needle guard 4 is still pressed against the injection site with the needle 2e inserted into the patient's skin and the activation sleeve 22 is in its most distal position relative to the housing 23. The activation sleeve biasing element 25 in a state in which it is biases the activation sleeve 22 (e.g. is a compression spring in a compressed state) and the (engaged-9 needle guard 4 in the proximal direction. When the user lifts/removes the needle guard 4 from the injection site, the biasing force exerted by the activation sleeve biasing element 25 causes the activation sleeve 22 and the (engaged) needle guard 4 to move proximally as indicted by the directional arrows in FIG. 26.

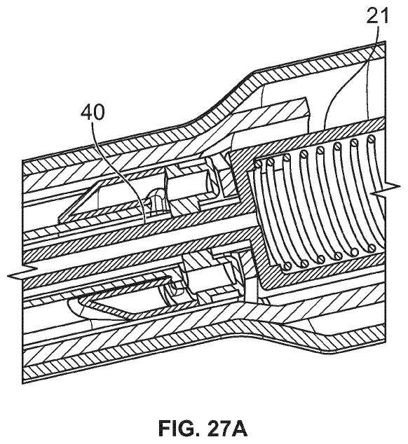

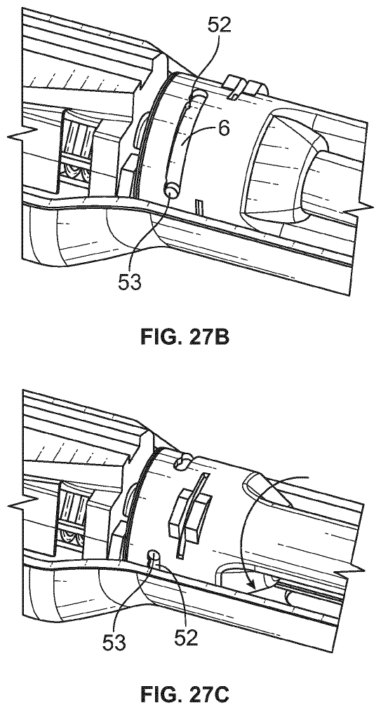

As indicated in FIG. 26, the needle guard 4 has moved to an extended position covering the injection needle 2e. At this stage of the injection process, the needle guard 4 is not prevented from being pushed back inside the housing 23. However, removal of the used disposal cassette subassembly 10 from the reusable drive mechanism subassembly 20 activates a locking mechanism that prevents axial movement of the needle guard 4 relative to the cassette body 3. Removal of the disposable cassette subassembly 10 requires that the user twist the cassette body 3 in a direction opposite to that which was used to attach the disposable cassette subassembly 10. This removal twisting causes the cassette body 3 to rotate relative to the needle guard 4, thus reengaging the axial locking mechanism described above and illustrated in FIGS. 5-7. The removal twisting also rotates the cassette body 3 relative to the plunger rod interface 6, which is now rotationally fixed relative to the plunger rod 21 because the inward radially protruding ribs 6a on the inside surface of the plunger rod interface 6 are engaged with longitudinal slot 40 on the outside surface of plunger rod 21. This engagement is illustrated in FIG. 27a and the effect of the removal twisting is illustrated in FIGS. 27b and 27c, where peg 53 protruding radially outward from the surface of plunger rod interface 6 reengages with the detent 52 thus preventing relative rotation between the cassette body 3 and the plunger rod interface 6.

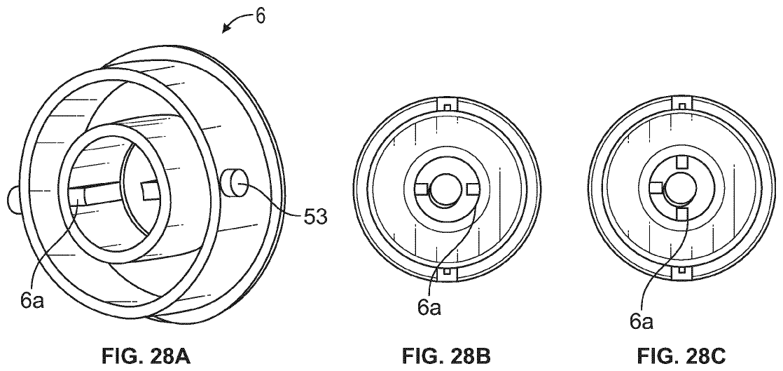

FIGS. 28a to 28c illustrate the positioning of the plunger rod interface 6, and specifically the protruding radial ribs 6a, on both 1) an unused, e.g. new, disposable cassette subassembly 10, e.g. before attachment to the reusable drive mechanism subassembly 20 (FIGS. 28b) and 2) a used disposable cassette subassembly 10, e.g. when attached to and after removed from the reusable drive mechanism subassembly 20 (FIG. 28C). It will be noted that the circumferential orientation of the radial ribs 6a on a used disposable cassette subassembly 10 is changed from the orientation of the new disposable cassette assembly 10. The orientation change prevents reuse of a used disposable cassette subassembly 10 because the radial ribs 6a are no longer in alignment with the bearing surface 46 on the plunger rod 21. Instead, the radial ribs 6a are aligned with the longitudinal slot 40, thus preventing the plunger rod interface 6 from forcing the plunger rod 21 distally to the cocked position.

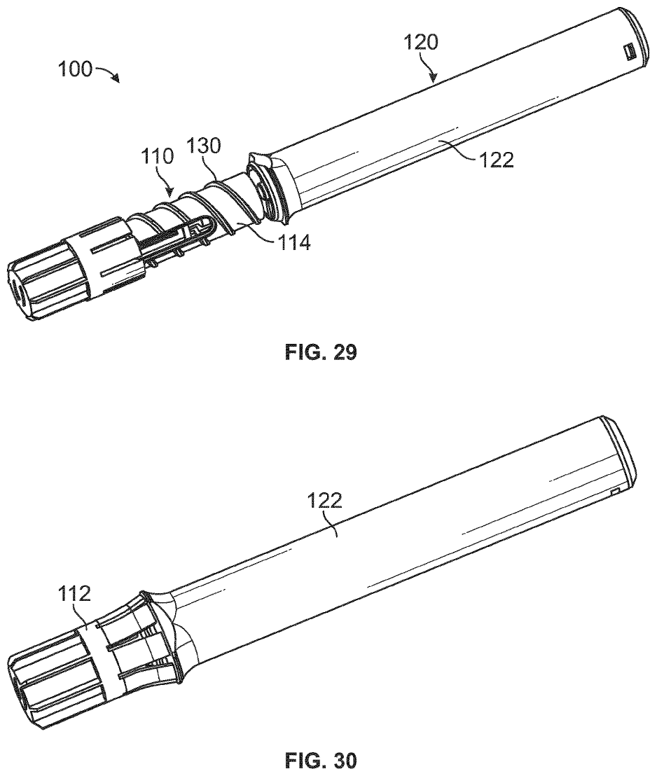

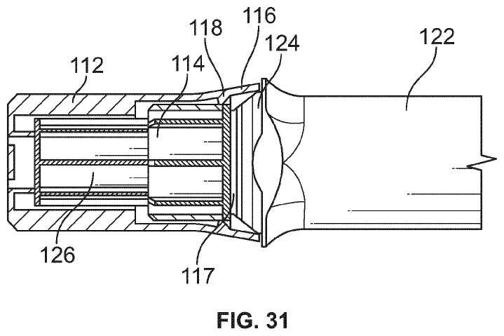

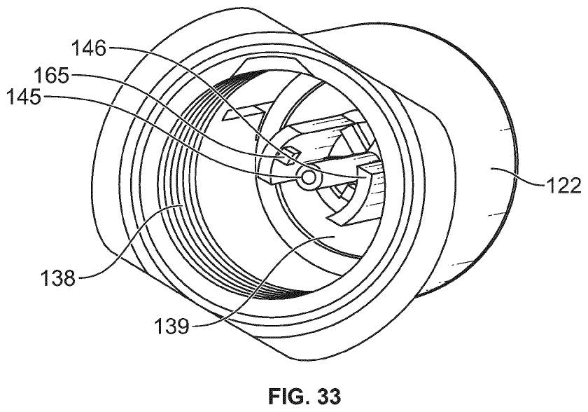

FIGS. 29-39 present another possible embodiment of a medicament delivery device 100 of the present disclosure, where a disposable cassette subassembly 110 is configured for releasable connection to reusable drive mechanism subassembly 120. The releasable connection can be a threaded attachment between a thread 130 on the outer surface of the cassette body 114 and a corresponding thread 138 on the inside surface of outer housing 122 (see FIG. 33). The outer housing 122 has a distal end having an end cap 139 that is secured to the housing by snap fit 140. Attached to a proximal surface of the end cap 139 is a guide rod 145 that is configured to fit into and support drive element 135. The end cap 139 also has support arms 146 that cooperate with the activation sleeve 131, e.g. by fitting into it, to guide axial movement of activation sleeve 131 during cocking/setting and activation of the medicament delivery device 100.

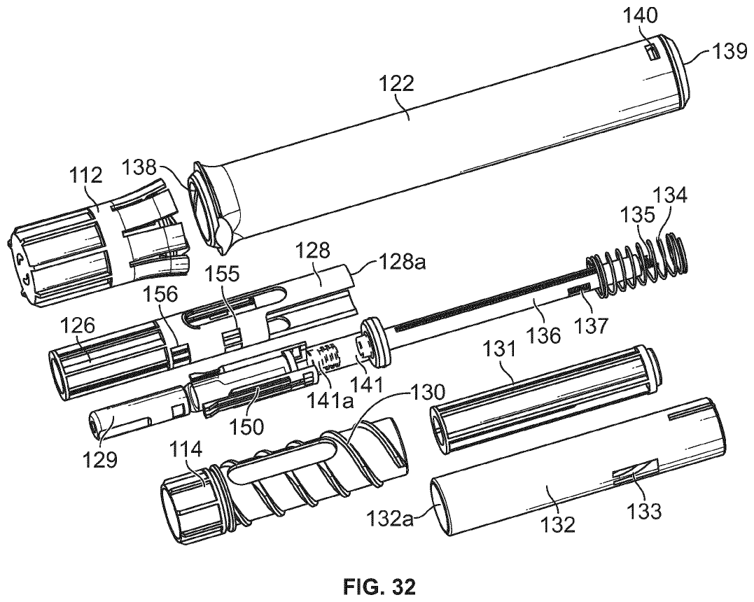

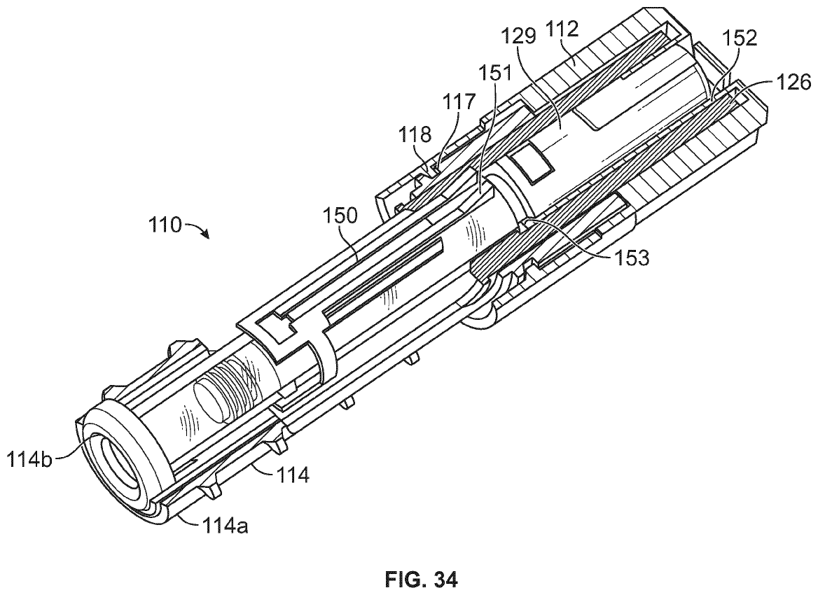

As with the first embodiment described above, the disposable cassette subassembly 110 includes a medicament container 141, shown in FIG. 32 as a pre-filled syringe, a needle shield assembly 129, a needle guard 126 and a protective cap 112. The cap 112 is releasably connected to the proximal end of cassette body 114, e.g. through a snap fit engagement of bead 118 located on the inside distal end of cap 112 with circular channel 117 located on the outside surface of cassette body 114 (see FIG. 34). Before the disposable cassette subassembly 110 is attached to the reusable drive mechanism subassembly 120, the engagement of the bead 118 with channel 117 prevents the cap 112 from being pulled off or otherwise removed from the proximal end of cassette body 114. When the disposable cassette subassembly 110 is screwed into the housing 122 eventually the proximal ends of segmented fingers 116 of cap 112 will engage ramp surface 124 of housing 122 (see FIGS. 30 & 31). This axial engagement of fingers 116 with ramp 124 will cause the fingers to splay radially outward, which in turn causes the bead 118 to disengage from channel 117. Once the fingers are splayed outward, the user can then pull the cap 112 proximally away from the cassette body 114. As the cap 112 is pulled, grip 153 on needle shield remover 152, which is axially fixed on the inside surface of the cap 112 (see FIG. 34) will engage with a distal end of needle shield assembly 129 thus simultaneously removing the needle shield assembly with the cap 112.

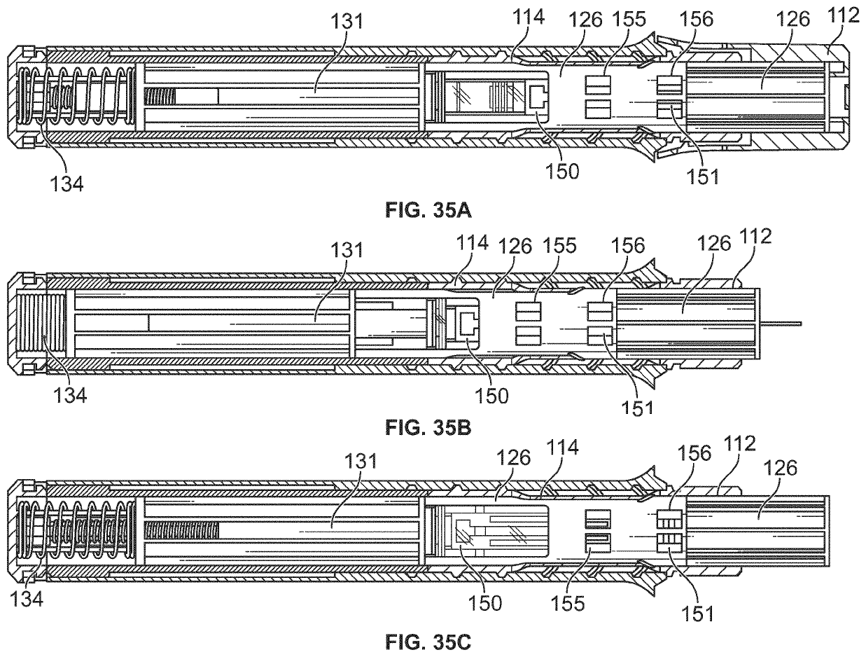

The disposable cassette subassembly 110 also can include a needle guard lock 150 having flexible fingers 151. FIGS. 35a to 35c illustrate three stages of the medicament delivery and specifically the interaction of the needle guard lock 150 with the needle guard 126. When the cap 112 is connected to the cassette body 114 and/or before the proximal end of the needle guard 126 is pressed against an injection site, the fingers 151 of needle guard lock 150 will be engaged with proximal cut-out 156. As illustrated in the second stage of operation in FIG. 35b, as the needle guard 126 is retracted into the housing 122 when pushed against an injection site, the distal axial movement of the needle guard 126 carries with it the needle guard lock 150 causing the distal end of the of the needle guard lock 150 to move axially to a distal locked position relative to the needle guard 126. Also in the second stage, the distal axial movement of the needle guard 126 pushes activation sleeve 131 distally causing activation sleeve biasing element 134 against its biasing force, e.g. to compress. In the third stage operation, where the user has removed the needle guard 126 from the injection site, the activation sleeve 131 and needle guard 126 are driven axially forward (proximally) by the biasing force of the activation sleeve biasing element 134. Because the needle guard lock 150 is axially fixed relative to the cassette body 114, the needle guard 131 will slide proximally relative to the needle guard lock 150 which causes the fingers 151 to disengage from proximal cut-out 156 and move into distal cut-out 155. The engagement between fingers 151 and distal cut-out 155 prevents the needle guard 126 from moving proximally relative to the cassette body 114.

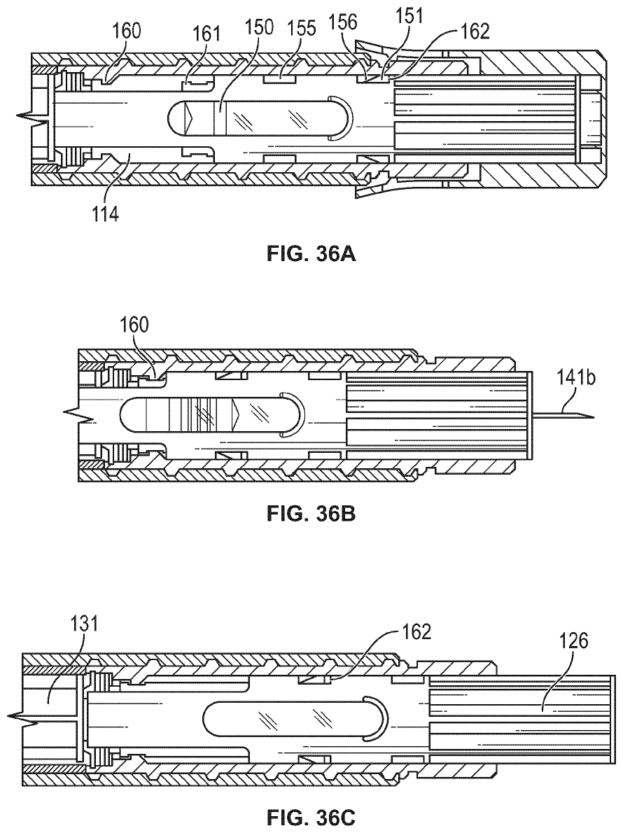

FIGS. 36a to 36c present a side view of the same three stages of operation that are shown in FIGS. 35a to 35c. Fingers 151 are wedged-shaped having a bearing surface 162 that engages with either cut-out 155 or cut-out 156 depending on the stage of operation. The distal end 161 of needle guard lock 150 is configured to engage with inward tab 160 on the cassette body 114 when the needle guard 126 is retracted distally relative to the cassette body 114 as illustrated in the second stage of operation (FIG. 36b). This engagement between tab 160 and distal end 161 of lock 150 will axially fix the needle guard lock 150 with the cassette body 114. As the proximal movement of the activation sleeve 131 pushes the needle guard 126 proximally during the third stage of operation, the fingers 151 will remain axially fixed as the needle guard slides proximally causing disengagement of the fingers 151 from cut-out 156 followed by engagement with cut-out 155. The bearing surface 162 then prevents the needle guard 126 from moving distally and thus covering the injection needle 141b to prevent accidental sticks.

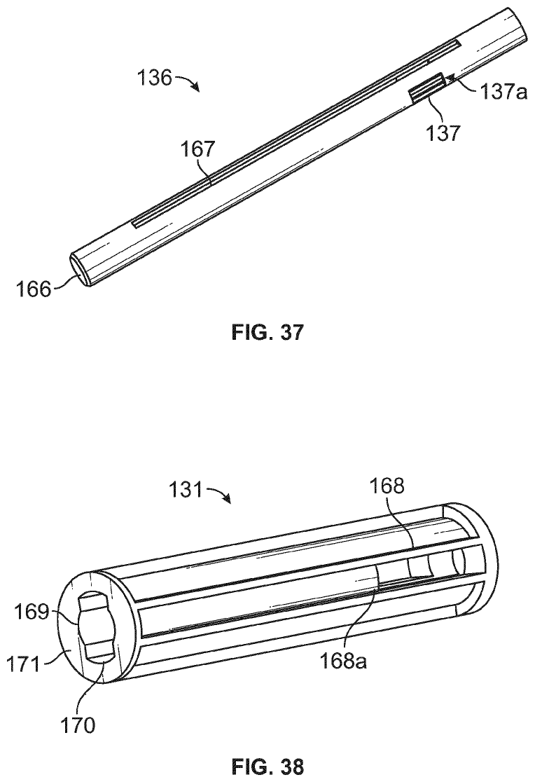

As with the above described first embodiment, the embodiment of the medicant delivery device 100 as illustrated in FIGS. 29-39 is cocked/loaded through the connection of the disposable cassette subassembly 110 with the reusable drive mechanism subassembly 120. As the disposable cassette subassembly 110 is screwed into housing 122, the terminal distal end 114a of cassette body 114 abuts terminal end face 132a of the inner housing 132 and pushes the inner housing 132 distally relative to the housing 122. Inward directed flexible arm 133 engages bearing surface 137a of the cut-out 137 located at the distal end of plunger rod 136 (see FIGS. 37 & 39). This engagement will cause the plunger rod 136 to also move distally with the inner housing 132 and will compress the drive element 135 which is partially positioned within the plunger rod 136 (which is hollow) where a proximal end of drive element 135 abuts the inside surface of proximal closed end 166 of plunger rod 136. Plunger rod 136 also has a longitudinal slot 167 that is in a guiding/sliding engagement with protrusion 165 (see FIG. 33) which is operatively associated with end cap 139 and support arms 146. Guide rod 145 is positioned within drive element 135 to prevent buckling of the drive element, which in this embodiment is a spring, or more specifically a helical compression spring. Support arms 146 have an arcuate shape and are slidably positioned within corresponding channel 170 (see FIG. 38) in the activation sleeve 131, which channel 170 has a semi-circular cross section 169 that accommodates and guides axial movement of the circular outside surface of plunger rod 136.

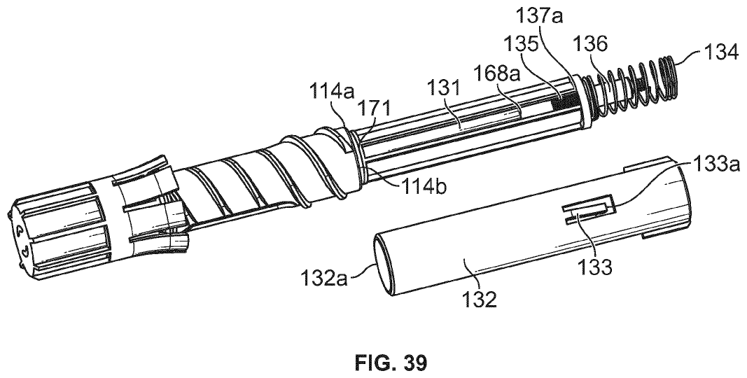

As the disposable cassette subassembly 110 is connected to the drive mechanism subassembly 120, distal facing surface 114b will engage with proximately facing surface 171 of the activation sleeve 131. This engagement causes the activation sleeve 131 to move distally simultaneously with the distal movement of the inner housing 132. Activation sleeve biasing element 134 is biased (e.g. compressed) by the distal axial movement of activation sleeve 131 (see FIG. 39). Once the disposable cassette subassembly 110 is fully connected to the reusable drive mechanism subassembly 120, the plunger rod 136 is locked and biased to move in the proximal direction. The user then removes protective cap 112 from the disposable cassette sub assembly 110, places the terminal proximal end of needle guard 126 against an injection site, and pushes the outer housing 122 proximally relative to the needle guard 126. This causes the needle guard 126 to retract distally into the cassette body 114, where the end surface 128a of the distally projecting arms 128 engages surface 171 of the activation sleeve 131, causing the activation sleeve 131 to move distally relative to the inner housing 132 such that bearing surface 168a in cut out 168 of the activation sleeve 131 contacts flexible arms 133 causing the flexible arms 133 to move radially outward such that end surface 133a disengages from bearing surface 137a of plunger rod 136. This disengagement of end surface 133a from 137a activates the medicament delivery device 110 by releasing the plunger rod 136 such that the drive element 135 pushes the plunger rod 136 proximally relative to the cassette body 114 and medicament container 141. The proximal end of the plunger rod 136 contacts and pushes the piston 141a proximally causing the medicament in the medicament container 141 to be discharged and administered through needle 141b.

After the medicament is delivered, the user removes the needle guard 126 from the injection site. This causes the compressed activation sleeve biasing element 134 to move the activation sleeve 131 and needle guard 126 proximally until the needle guard 126 is locked into its final extended position by needle guard lock 150, as described above. The disposable cassette subassembly 110 can now safely be removed from the drive mechanism subassembly 120 by twisting/screwing the disposable cassette subassembly 110 in a direction opposite to that used to connect the disposable cassette subassembly 110 to the drive mechanism subassembly 120. As the disposable cassette subassembly 110 is being disconnected from the drive mechanism subassembly 120, further biasing force of activation sleeve biasing element 134 causes both the inner housing 132 and activation sleeve 131 to move proximally back to their initial starting positions. This final axial movement will also cause end surface 133a to reengage with surface 137a of the plunger rod 136. The reusable drive mechanism assembly 120 is now ready to receive a new/unused disposable cassette subassembly 110.

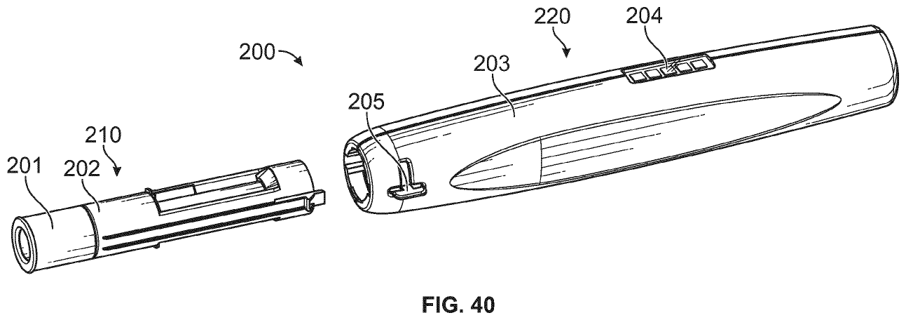

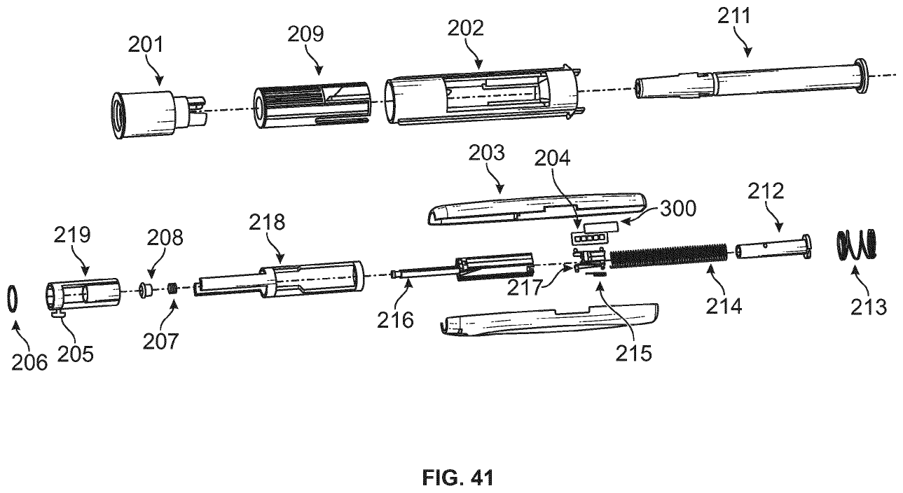

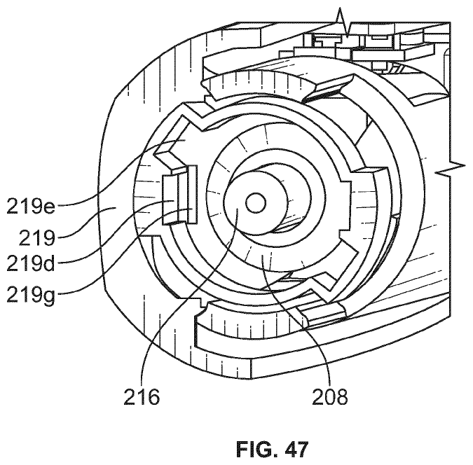

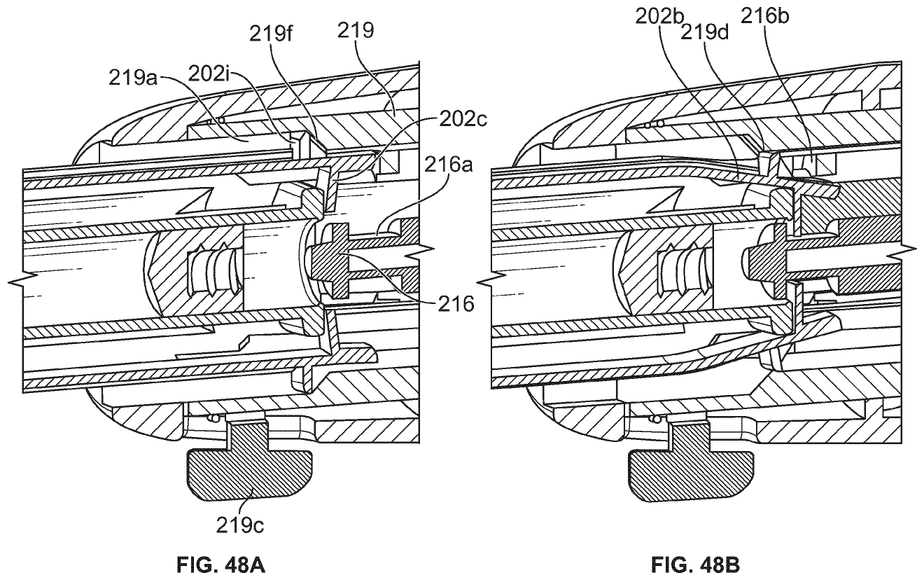

Yet another embodiment of the present disclosure is illustrated in FIGS. 40-56b. This medicament delivery device 100 is similar in construction and operation to the above-described first embodiment. Differences include the presence of an electronics module 204 in the reusable drive mechanism subassembly 220 and the use of a spring clip connection between the distal end of disposable cassette subassembly 210 and the proximal end opening of the reusable drive mechanism subassembly 220. FIG. 41 presents an exploded view of each of the subassemblies 210, 220 of this third embodiment of the medicament delivery device 200. As with the above-described embodiments, the disposable cassette subassembly 210 includes a needle guard 209 that slides relative to cassette body 202 and axially locks relative to housing 203 in an extended position when the user removes the terminal proximal end of the needle guard 209 from an injection site after medicament delivery.

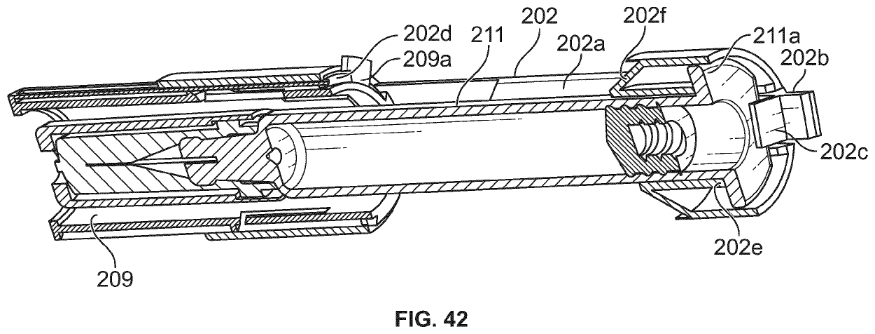

FIG. 42 illustrates a cross-sectional view of the interaction of the medicament container 211 with the cassette body 202. Flexible arm 202b, with transverse flange 202c, is initially splayed radially outward to allow for the insertion of medicament container 211 into the distal opening of the cassette body 202. Medicament container 211 is slid proximally relative to the cassette body 202 until the proximal side of finger flange 211a abuts a stop surface. Flexible arm 202b is than allowed to flex back radially inward such that the transverse flange 202c abuts the distal side of finger flange 211a of the medicament container 211 to axially secure the medicament container 211 from movement relative to the cassette body 202. The cassette body 202 also has container guides 202a that assist with aligning the medicament container 211 within the cassette body 202 during assembly of the disposable cassette subassembly 210. Likewise, tapered section 202f constricts/reduces the inner diameter of the cassette body 202 to the approximate outer diameter of the medicament container 211 to further assist with alignment and insertion of the medicament container 211.

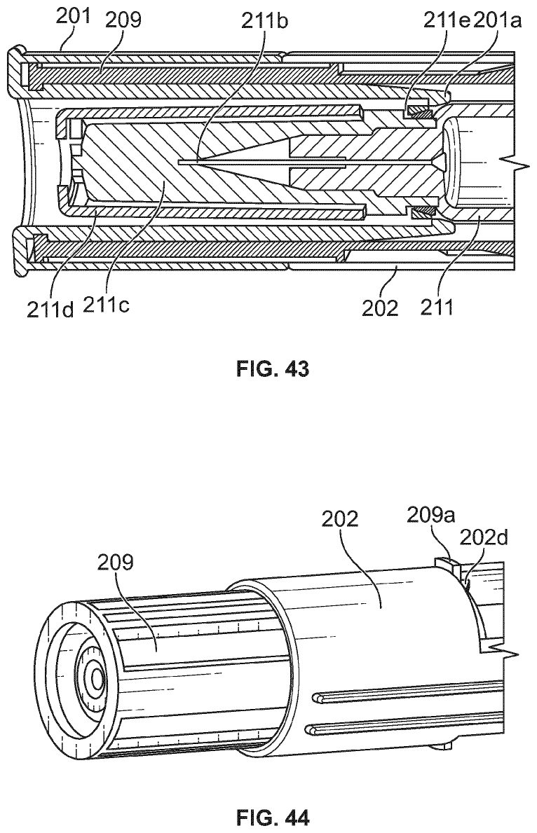

FIG. 43 shows a cross-sectional view of the protective cap 201 when attached to the cassette body 202. Cap 201 has a U-shaped configuration that accommodates and surrounds the proximal end portion of needle guard 209. A distal end of the cap 201 terminates in gripper 201a that is configured to engage with a distal end of rigid needle shield 211d which is operatively connected with flexible needle shield 211c that protects and seals injection needle 211b. Once the disposable cassette subassembly 210 is connected to the reusable drive mechanism assembly 220, the user may pull cap 201 away from the proximal end of cassette body 202. As the cap 201 is pulled away, grip 201a engages with the rigid needle shield 211d causing the both the rigid and flexible needle shields 211d, 211c to move with the cap 201 and be removed from the proximal end of the medicament container 211. This leaves the needle 211b exposed but within and surrounded by needle guard 209.

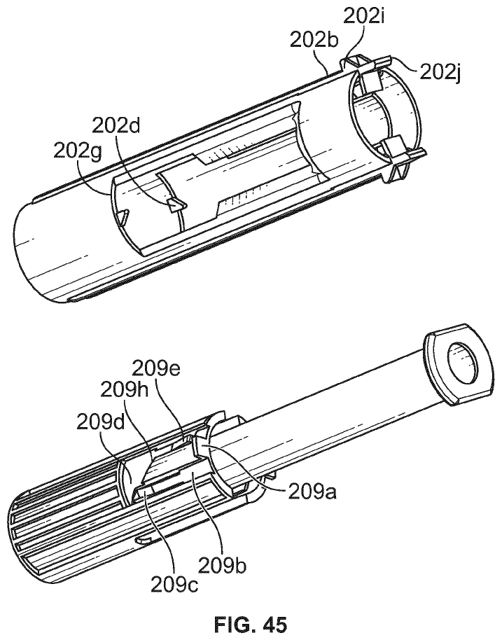

FIGS. 44-45 illustrate the relationship between the needle guard 209 and the proximal portion of the cassette body 202. An outward extending tab 209a engages a distal projecting surface of cut-out 202g to prevent the needle guard 209 from moving axially relative to the cassette body 202 when the needle guard 209 is in its fully extend position before and after medicament delivery. The outer surface of the needle guard 209 also contains guide channels 209b, 209d configured to accept protrusion 202d during axial movement of the needle guard 209 relative to the cassette body 202. When the terminal proximal end of the needle guard 209 is pushed against an injection site, the needle guard 209 will move distally relative to the cassette body 202 causing the protrusion 202d to move within channel 209b until it engages with and rides over detent 209c, which prevents the protrusion from reentering channel 209b. When the medicament delivery is complete and the user removes the needle guard 209 from the injection site, the activation sleeve biasing element 213 will cause the needle guard 209 to move proximally. As the needle guard 209 is pushed proximally to its final extended proximal position, the angled edge of protrusion 202d will act as a cam follower as it traverses channel 209d and angled surface 209h causing a slight rotation of the needle guard 209 relative to the cassette body 202. As the needle guard 209 rotates and moves proximally the protrusion 202d will move along channel 209d until it engages and moves up and over guard lock 209e. Once the protrusion 202d is positioned distally relative to the guard lock 209e, the needle guard 209 is prevented from distal movement, e.g. retraction, relative to the cassette body 202.

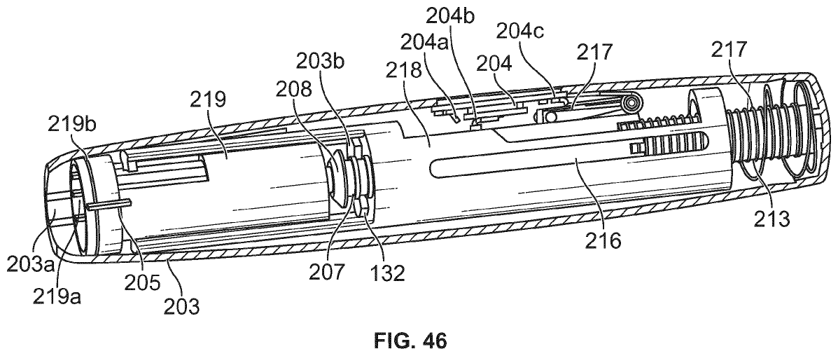

Unlike the required twisting motion needed in the two previously mentioned embodiments of the present disclosure, the connection of the disposable cassette subassembly 210 with the reusable drive mechanism subassembly 220 requires only an axial alignment and linear insertion of the distal end of the cassette body 202 with cassette interface 219 which is axially fixed to but rotatably positioned within and relative to housing 203. In other words, this third embodiment does not require a relative twisting/turning of the disposable cassette subassembly 210 relative to the outer housing 203 of the drive mechanism subassembly 220. Flexible arm 202b is aligned with housing slot 203a and channel 219a inside cassette interface 219 (see FIGS. 46-49b). Lip 202i on flexible arm 202b will slide within channel 219a until it engages ramp 219f which causes flexible finger 202b to flex radially inward such that flange 202c is forced into a reduced diameter section 216a of plunger rod 216. As the cassette body 202 is pushed further distally, a distal surface of flange 202c will abut and engage bearing surface 216b pushing plunger rod 216 distally. As described above in the first embodiment, the distal movement of the plunger rod 216 continues until plunger lock 217 engages the plunger rod 216 to prevent compressed spring 214 from moving the plunger rod 216 proximally. The plunger rod 216 is now cocked/loaded and prevented from moving proximally by the plunger rod lock 217. The plunger rod lock 217 is configured and operates in basically the same manner as previously described in the first embodiment.

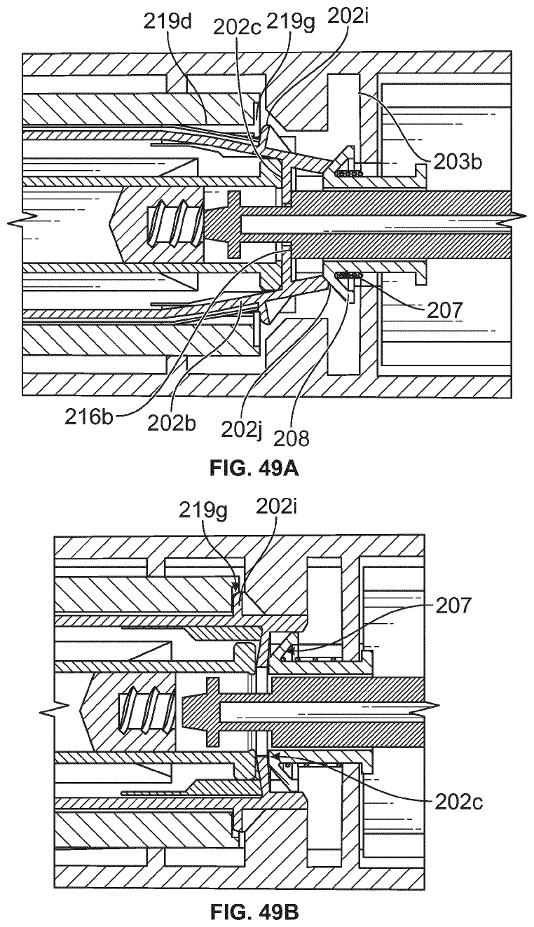

FIGS. 49a and 49b illustrate the positioning of the distal end of the cassette body 202 just before the disposable cassette subassembly 210 is fully connected to the reusable drive mechanism sub assembly 220 and the plunger rod 216 is fully cocked and locked. As described above, as the cassette body 202 is pushed distally with the flexible finger 202b being forced to flex radially inward by channel 219d such that flange 202c pushes on bearing surface 216b of plunger rod 216 to move it distally to the fully cocked/loaded position. Terminal distal end 202j of flexible arm 202b engages with cassette lock 208 pushing it distally and compressing cassette lock biasing element 207 (which is, e.g., a spring). Cassette lock 208 is guided by and slidably positioned within housing web 203b. Once lip 202i reaches the end of channel 219d, the flexible arm springs radially outward such that lip 202i snaps into stop face 219g. Because the plunger rod 216 is now locked axially through engagement with plunger rod lock 217, the flange 202c is no longer engaged with bearing surface 216b. Cassette lock 208 will be forced proximally by cassette lock biasing element 207 to bias flange 202c against the medicament container 211 locking it from axial movement and to prevent the flange 202c from bending back radially inward into section 216a of the plunger rod 216. The radially outward movement of the flange 202c also clears an axial path for the plunger rod 216 to move proximally when the medicament delivery device 200 is activated to dispense the medicament in the medicament container 211.

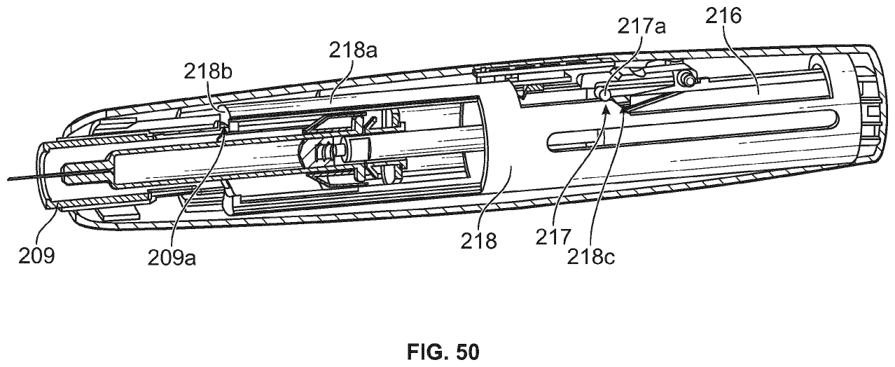

FIG. 50 illustrates the medicament delivery device 200 as it is activated when the needle guard 209 is pushed against an injection site. Outward extending tab 209a engages and pushes on the terminal end 218b of arms 218a of activation sleeve 218 causing it to slide distally relative to the housing 203. This distal movement also causes ramped surface 218c to engage guide pin 217a lifting the plunger lock 217 from locking engagement with the plunger rod 216 as explained above in connection with the first embodiment. The plunger rod 216 is then driven forward proximally by the decompression of plunger rod biasing element 214.

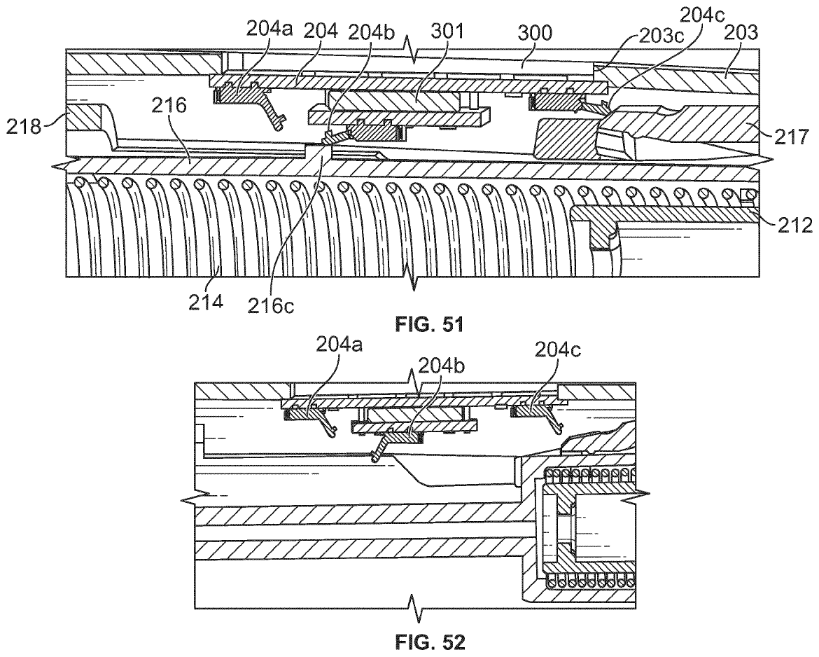

As mentioned, this third embodiment may also contain an electronic unit 204 that is used to monitor the operation of the of medicament delivery device 200 (see FIG. 41) through relative movement of a plurality of sensors, such as, switches or other mechanical contact electrical components. FIGS. 51-55 illustrate three configurations of a needle guard switch 204a, a plunger rod switch 204b, and a plunger rod lock switch 204c that can form part of the electronic unit of 204 which is mounted to an inside surface of housing 203 such that a light guide 300 can be viewed by a user of the medicament delivery device 200 through a cut-out 203c in the housing 203. The electronic unit 204 can be powered by battery 301, which may be rechargeable and/or replaceable. FIG. 51 depicts the electronic unit 204 prior to the plunger rod 216 being cocked/loaded, where switch 204a is in an “out” position and not engaged with needle guard 209, switch 204b is in an “in” position being engaged with radial nib 216c on the outside surface of plunger rod 216, and switch 204c is in an “in” position being engaged the plunger rod lock 217. The electronic unit 204 can be configured to provide the user of the status of the medicament delivery device 200 as a function of the relative positions of the switches 204a-c. Such user notification can be viewed as light seen through light guide 300. Notification could also be provided through sound and/or tactile signals generated by the electronic unit 204. In this initial state the electronics are in a sleep mode. When the patient mounts the disposable cassette sub-assembly 210, the cassette switch 204b activates the electronics unit 204 and switch 204c confirms a cassette loaded state. Notifications to the user may be audible, tactile or visible to confirm a successful cassette loading (204b and 204c changes state) or unsuccessful (no state change on switch 204). Notifications may also be provided in a smart device application/program. Data may be stored locally in the electronic unit 204 on a storage memory and/or transmitted e.g., via Bluetooth Low Energy (BLE) to a smart device.

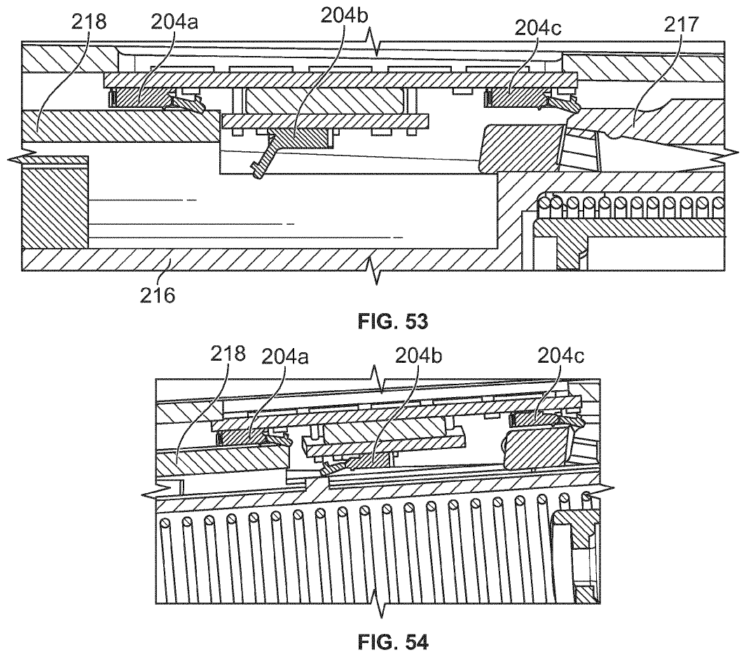

FIG. 52 depicts the configuration of the switches 20aa, 204b, 204c after the plunger rod 216 has been cocked and locked, i.e., after the disposable cassette subassembly 210 has been fully connected to the reusable drive mechanism subassembly 220. Nib 216c is no longer engaged with switch 204b which has now moved to an “out” position. Switch 204c is no longer engaged with plunger lock 217 and so it also has moved to an “out” position. FIG. 53 depicts the configuration of the medicament delivery device 200 after activation where the plunger lock 217 has disengaged from the plunger rod 216 and has contacted switch 204c moving it to the “in” position. The activation sleeve 218 has been moved distally by the retraction of the needle guard 209 into the housing 203 and has engaged with switch 204a moving it to the “in” position. Switch 204b remains in the “out” position. Switch 204a is activated when the user pushes the medicament delivery device 200 towards the body to start the injection. This switch 204a also monitors that the patient maintains the needle guard 209 pressed against the injection site throughout the full injection process. If the patient lifts the needle guard 209 before a full injection has been delivered, this information may be stored locally within the electronic unit 204 or can be transmitted to a BLE receiver. A health care provider and/or the patient may be notified so as to help the user of the medicament delivery device 200 to perform correct injections. A calculation of delivered partial dose may be performed by comparing the time for the dose delivery, i.e., the time between pushing 204c inwards to 204b being pushed inwards, and the injected dose, i.e., the time between 204c being pushed inwards to 204a being pushed outwards. Switch 204a may also start an injection feedback signal to the user (audible, tactile or visual), the feedback could end after the full dose is given, i.e., 204b pushed inwards, with an additional holding time to let the drug be absorbed in the tissue before lifting the medicament delivery device 200.

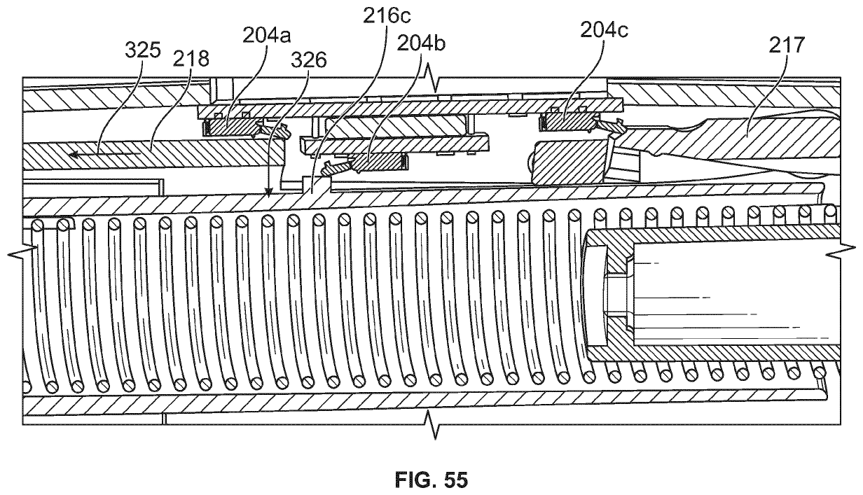

FIG. 54 illustrates the switch positions upon completion of medicament delivery from the medicament container 211, but prior to the user lifting the needle guard 209 from the injection site, i.e., the needle guard 209 is still in its most distal position relative to housing 203. In this phase of device operation, the activation sleeve remains engaged with switch 204a, the nib 216c has reengaged with switch 204b and plunger lock 217 remains engaged with switch 204c. In other words, all switches 204a, 204b, 204c are in the “in” position. Once the user removes the disposable cassette subassembly 210, the switches 204a, 204b, 204c will return to the configuration illustrated in FIG. 51. The electronic unit 204 can also monitor whether the user is correctly operating the medicament delivery device 200. For example, FIG. 55 illustrates the situation where the user has prematurely removed/lifted the medicament delivery device 200 from an injection site before delivery of the medicament is complete. Such an improper removal from the injection site will cause the activation sleeve 218 to move proximally by the force of the activation sleeve biasing element 213, as indicated by directional arrow 325. As the activation sleeve 218 moves proximally, the needle guard switch 204a will disengage from the activation sleeve 218 moving to the “out” position, as indicated by directional arrow 326. This occurs before needle guard switch 204b is moved to the “in” position by contact with nib 216c on the plunger rod. Electronic unit 204 would then provide a signal to the user of the incomplete/improper delivery of the medicament.

In each phase of operation or configuration of the delivery device 200 of the third embodiment, the electronic unit 204 can notify the user of the status of the device. Such data can be stored by the electronic unit, transmitted (wired or wirelessly) to a remote location, or transferred to another part of the device or add-on component attached to the device, for example, utilizing RFID tags or the like transmitters/receivers.

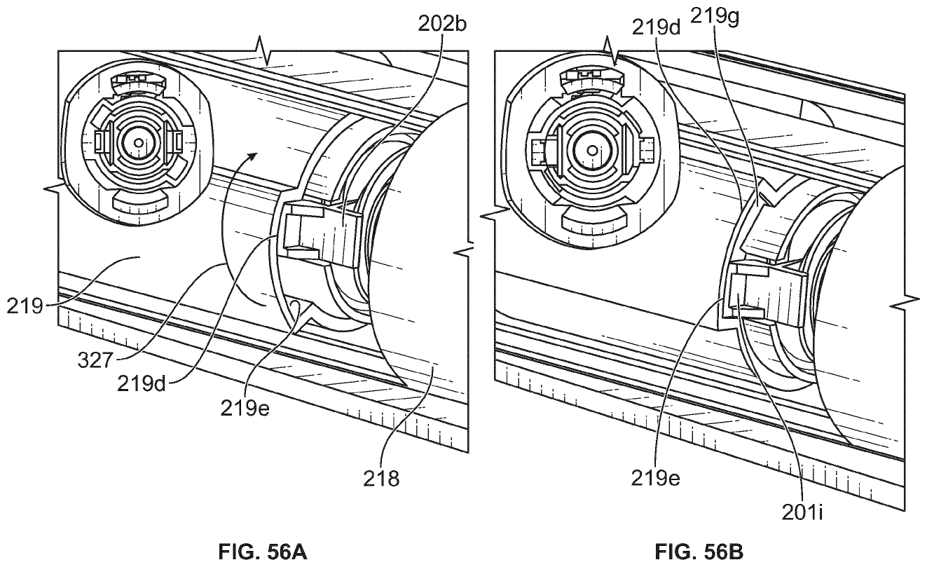

Once the delivery of medicament is complete and the needle has been removed from the injection site, the disposable cassette subassembly 210 can be removed from the reusable drive mechanism subassembly 220. To disengage the cassette body 202 from the 2219, the user must manipulate cassette release lever 205 which causes rotation of the cassette interface 219 relative to the cassette body 202 and overcomes the circumferential biasing force exerted on the cassette interface 219 by interface biasing element 206. FIGS. 56a and 56b illustrate the “before” and “after” alignment of the cassette interface with finger 202b where directional arrow 327 indicated the direction of rotation of the cassette interface 219 when the user moves lever 205 relative to housing 203. Rotation of the interface 219 changes the alignment between finger 202b and channels 219d and 219e. Aligning finger 202b with channel 219e removes stop face 219g from its blocking function and allows lip 202i to slide freely within channel 219e. The disposable cassette subassembly 210 can now be pull axially out (proximally) from housing 203. A new disposable cassette subassembly 210 can then be loaded (connected) to the reusable drive mechanism subassembly 220 for dispensing medicament from a new (fresh) medicament container 211.

Whilst the invention is illustrated above by some specific embodiments, it will be appreciated that elements and components of the described embodiments may be interchanged with one another to provide further such embodiments. In short, the features of the different embodiments may be mixed or combined as appropriate.

1-29. (canceled)

30. A medicament injection device comprising:
a reusable drive mechanism subassembly comprising:
a tubular housing having a proximal end and a distal end;
a plunger rod comprising a proximal portion and a distal portion;
a drive element at least partially positioned in a hollow section of the distal portion of the plunger rod; and
a plunger rod lock pivotally attached to the tubular housing, biased radially with respect to the length of the tubular housing and being engageable with the plunger rod; and
a disposable cassette subassembly comprising:
a cassette body arranged to receive a medicament container having an injection member;
a needle guard at least partially positioned inside and slidable relative to the cassette body; and
a plunger rod interface positioned at a distal end of the cassette body such that the cassette body can rotate relative to the plunger rod interface,
wherein the plunger rod interface engages with the proximal portion of the plunger rod when the disposable cassette subassembly is operatively connected to the reusable drive mechanism subassembly, and pushes the plunger rod distally until the plunger rod lock transitions from a released position to a locked position in which the plunger rod is prevented from proximal axial movement relative to the cassette body.

31. The medicament injection device of claim 30, wherein the cassette body has a locking hub protruding radially from an outside surface and configured to form a releasable bayonet attachment with an inside surface of the tubular housing.

32. The medicament injection device of claim 30, wherein the needle guard has a distally projecting tab for engaging a proximal end of the activation sleeve to move the activation sleeve distally during activation of the medicament injection device.

33. The medicament injection device of claim 30, further comprising:
an activation sleeve at least partially surrounding the plunger rod and slidable relative to the tubular housing; and
an activation sleeve biasing element that biases the activation sleeve in a proximal direction relative to the tubular housing.

34. The medicament injection device of claim 33, wherein the needle guard is rotationally fixed relative to the activation sleeve when the disposable cassette subassembly is operatively connected to the reusable drive mechanism subassembly.

35. The medicament injection device of claim 33, wherein the cassette body rotates relative to the needle guard when the disposable cassette subassembly is operatively connected to the drive mechanism subassembly.

36. The medicament injection device of claim 35, wherein the needle guard is in an axially fixed state relative to the cassette body prior to the rotation of cassette body relative to the needle guard, and wherein rotation of the cassette body during connection of the disposable cassette subassembly to the reusable drive mechanism subassembly transitions the needle guard to a distally movable state such that the needle guard engages with and moves the activation sleeve distally.

37. The medicament injection device of claim 36, wherein the distal movement of the activation sleeve causes the activation sleeve to engage the plunger rod lock moving the plunger rod lock from a locked position to a released position releasing the plunger rod to move proximally relative to the cassette body for dispensing medicament from the medicament container.

38. The medicament injection device of claim 30, wherein ribs on an inside surface of the plunger rod interface engage with a bearing surface on the proximal portion of the plunger rod to push the plunger rod distally to the locked position.

39. A two-part medicament injection device comprising:
a reusable drive mechanism subassembly comprising:
a tubular housing having an open proximal end and a closed distal end;
a plunger rod comprising a proximal portion having a first diameter and a distal portion having a second diameter that is larger than the first diameter;
a drive spring partially positioned in a hollow section of the distal portion;
an activation sleeve partially surrounding the plunger rod and slidable relative to the tubular housing; and
a plunger rod lock mechanism comprising a radially biased plunger rod lock pivotally attached to the housing such that the plunger rod lock operatively engages with the plunger rod and the activation sleeve; and
a disposable cassette subassembly comprising:
a cassette body;
a cap removably attached to the cassette body;
a needle guard partially positioned inside and slidable relative to the cassette body, where the cassette body rotates relative to the needle guard when the disposable cassette subassembly is operatively connected to the drive mechanism subassembly;
a plunger rod interface positioned at a distal end of the cassette body such that the cassette body can rotate relative to the plunger rod interface; and
a medicament container comprising an attached needle,
wherein the plunger rod interface engages with the proximal portion of the plunger rod when the cassette subassembly is connected to the drive mechanism subassembly pushing the plunger rod distally until the plunger rod lock mechanism transitions from a released position to a locked position such that the plunger rod is prevented from proximal axial movement relative to the medicament container.

40. The injection device of claim 39, further comprising an activation sleeve spring that biases the activation sleeve in a proximal direction relative to the tubular housing.

41. The injection device of claim 39, wherein the cassette body has a locking hub protruding radially from an outside surface and configured to form a releasable bayonet attachment with an inside surface of the tubular housing.

42. The injection device of claim 39, wherein a distally projecting tab on the needle guard engages a proximal end of the activation sleeve to move the activation sleeve distally during activation of the injection device.

43. The injection device of claim 10, wherein the needle guard is rotationally fixed relative to the activation sleeve when the disposable cassette subassembly is operatively connected to the reusable drive mechanism subassembly.

44. The injection device of claim 39, wherein the needle guard is in an axially fixed state relative to the cassette body prior to the rotation of cassette body relative to the needle guard, where rotation of the cassette body during connection of the cassette subassembly to the drive mechanism subassembly will transition the needle guard to a distally movable state such that the needle guard will engage with and move the activation sleeve distally.

45. The injection device of claim 44, wherein the distal movement of the activation sleeve will cause the activation sleeve to engage the plunger rod lock moving the plunger rod lock from a locked position to a released position releasing the plunger rod to move proximally relative to the medicament container to dispense medicament.

46. The injection device of claim 44, wherein ribs on an inside surface of the plunger rod interface engage with a bearing surface on the proximal portion of the plunger rod to push the plunger rod distally to the locked position.

47. A two-part medicament injection device comprising:
a reusable drive mechanism subassembly comprising:
a tubular housing having an open proximal end and a closed distal end;
a plunger rod comprising a proximal portion having a first diameter and a distal portion having a second diameter that is larger than the first diameter;
a drive spring partially positioned in a hollow section of the distal portion;
an activation sleeve partially surrounding the plunger rod and slidable relative to the tubular housing; and
a plunger rod lock mechanism comprising a radially biased plunger rod lock pivotally attached to the housing such that the plunger rod lock operatively engages with the plunger rod and the activation sleeve; and
a disposable cassette subassembly comprising:
a cassette body comprising a distal end having a flexible finger;
a cap removably attached to the cassette body;
a needle guard partially positioned inside and slidable relative to the cassette body, where the needle guard rotates relative to the cassette body when moving from a retracted position to an extended position; and
a medicament container comprising an attached needle,
wherein the flexible finger engages with the proximal portion of the plunger rod when the cassette subassembly is axially inserted into and connected to the drive mechanism subassembly such that the plunger rod is pushed distally until the plunger rod lock mechanism transitions from a released position to a locked position to prevent proximal axial movement of the plunger rod relative to the medicament container.

48. The injection device of claim 47, wherein the reusable drive mechanism subassembly further comprises a cassette interface that is rotatable relative to the tubular housing.

49. The injection device of claim 48, wherein the cassette interface has a first longitudinal channel that is slidably engaged with the flexible finger to bias the finger radially inward to engage the proximal portion of the plunger rod.

50. The injection device of claim 49, wherein the cassette interface has a second longitudinal channel that aligns and becomes slidably engaged with the flexible finger when a release lever is moved to overcome a rotational spring bias of the cassette interface during disconnection of the disposable cassette subassembly from the reusable drive mechanism subassembly.

51. The injection device of claim 50, further comprising an electronics unit having a plurality of mechanical contact sensors that determined operational status of the injection device.

52. A two-part medicament injection device comprising:
a reusable drive mechanism subassembly comprising:
a tubular housing having an open proximal end and a closed distal end, where an inner surface of the proximal end comprises a first thread;
a hollow plunger rod comprising a closed proximal end and an open distal end, a drive spring partially positioned inside plunger rod;
an activation sleeve partially surrounding the plunger rod and slidable relative to the tubular housing, where a distal portion of the activation sleeve has a cut-out aligned with a cut-out in the plunger rod prior to activation of the injection device; and
an inner housing partially surrounding the activation sleeve and having a plunger rod lock mechanism comprising a flexible finger that releasably engages with the cut-out in the plunger rod; and
a disposable cassette subassembly comprising:
a cassette body comprising an outer surface comprising a second thread that matches and cooperates with the first thread such that the disposable cassette subassembly can be removably connected to the drive mechanism subassembly;
a cap rotationally fixed and removably attached to the cassette body;
a needle guard partially positioned inside and slidable relative to the cassette body, where the cassette body is rotationally fixed relative to the needle guard;
a needle guard lock that is slidable relative to the cassette body from a first position to a second position; and
a medicament container comprising an attached needle,
wherein axial distal movement of the needle guard moves the needle guard lock from the first position to the second position,
wherein a distal end of the cassette body engages a proximal end of the inner housing when the cassette body is being threadedly connected to the tubular housing causing the inner housing to move axially such that the flexible finger on the inner housing engages the cut-out in the plunger rod pushing the plunger rod distally and compressing the drive spring.

53. The injection device of claim 52, wherein the activation sleeve moves relative to the inner housing during activation of the injection device.

54. The injection device of claim 52, wherein movement of the activation sleeve distally relative to the inner housing causes the flexible finger to disengage from the cut-out in the plunger rod.

55. The injection device of claim 52, wherein the needle guard comprises a first cut-out and a second cut-out, where locking fingers at a proximal end of the needle guard lock are engaged with the first cut-out when the needle guard lock is in the first position and in the second position.

56. The injection device of claim 55, wherein axial proximal movement of the needle guard relative to the needle guard lock when in the second position causes disengagement of the locking fingers from the first cut-out and then engagement with the second cut-out.

57. The injection device of claim 52, wherein the closed distal end comprises an endcap having a longitudinal support that is positioned within the drive spring and support arms slidably engaged with an inside surface of the activation sleeve.

58. The injection device of claim 52, wherein the cap comprises a plurality of segmented fingers having inner surfaces configured with a snap fit connector that engages with a proximal end of the cassette body, where when the cassette body is threadedly connected to the tubular housing the segmented fingers will engage a ramp surface on the cassette body causing the segmented fingers to bend radially outward disconnecting the snap fit connector from the cassette body.
